(19) United States
(12) Patent Application Publication
(10) Pub. No.: US 2009/0041810 A1
(43) Pub. Date: Feb. 12, 2009

(54) Immunostimulating polyphosphazene compounds for intradermal immunization

(76) Inventors: Alexander K. Andrianov, Belmont, MA (US); Daniel P. Decollibus, Holliston, MA (US); Helice A. Gillis, North Attleboro, MA (US); Henry Hy Kha, Norwood, MA (US); Alexander Marin, Newton, MA (US)

Correspondence Address: Raymond E. Stauffer;c/o Carella, Byrne, Bain, Gilfillan,, Cecchi, Stewart & Olstein, 5 Becker Farm Road, Roseland, NJ, 07068, US

(21) Appl. No.: 12/217,402

(22) Filed: Jul. 3, 2008

Related U.S. Application Data
(60) Provisional application No. 61/125,576, filed on Apr. 25, 2008.
(60) Provisional application No. 60/948,540, filed on Jul. 9, 2007.

(51) Int. Cl. A61K 9/00; A61K 39/00; A61P 37/02

(52) U.S. Cl. 424/400; 424/184.1

(57) ABSTRACT

Disclosed are intradermally administered products and methods for producing an immune response in a human or in an animal comprising an antigen and a polyphosphazene polyelectrolyte adjuvant in an amount effective to elicit an immune response in the human or in the animal against said antigen.

This application claims priority to U.S. Provisional Application Ser. No. 61/125,576, filed Apr. 25, 2008; and also claims priority to U.S. Provisional Application Ser. No. 60/948,540, filed Jul. 9, 2007; the disclosures of each of which are hereby incorporated by reference in their entireties.

This invention is directed to a product comprising an antigen and an adjuvant capable of eliciting an immune response, in a human or in an animal, against the antigen; and it is further directed to methods of using an antigen and an adjuvant to elicit an immune response, in a human or in an animal, against the antigen.

This invention is particularly directed to an intradermally administered product comprising an antigen and an adjuvant capable of eliciting an immune response, in a human or in an animal, against the antigen; and it is further particularly directed to methods of using an antigen and an adjuvant to elicit an immune response, in a human or in an animal, against the antigen, by intradermally administering the antigen and the adjuvant to the human or to the animal.

This invention is more particularly directed to an intradermally administered product comprising an antigen and an adjuvant comprising a polyphosphazene polyelectrolyte capable of eliciting an immune response, in a human or in an animal, against the antigen; and it is further more particularly directed to methods of using an antigen and an adjuvant comprising a polyphosphazene polyelectrolyte to elicit an immune response, in a human or in an animal, against the antigen, by intradermally administering the antigen and the adjuvant comprising a polyphosphazene polyelectrolyte to the human or to the animal.

U.S. Pat. No. 5,494,673 discloses polyphosphazene polyelectrolytes that are useful as immunoadjuvants. The disclosures of U.S. Pat. No. 5,494,673 are hereby incorporated by reference in their entireties. Particular attention is directed to those sections of U.S. Pat. No. 5,494,673 titled “SUMMARY OF THE INVENTION,” “DETAILED DESCRIPTION OF THE INVENTION,” “SELECTION OF POLYPHOSPHAZENE POLYELECTROLYTES,” “SYNTHESIS OF PHOSPHAZENE POLYELECTROLYTES,” “SELECTION OF AN ANTIGEN,” “PREPARATION OF AN IMMUNOGENIC COMPOSITION,” “POLYMER—ANTIGEN CONJUGATES,” “CROSS-LINKED POLYMER ADJUVANT,” “ADDITIVES TO THE POLYMER—ADJUVANT MIXTURE,” and “ADMINISTRATION OF POLYMER—ANTIGEN—VACCINE,” and the subject matters disclosed thereunder are hereby incorporated by reference in their entireties. Particular attention is directed to those sections of U.S. Pat. No. 5,494,673 titled “EXAMPLES 1-10,” and the subject matters disclosed thereunder are hereby incorporated by reference in their entireties. Particular attention is directed to those sections of U.S. Pat. No. 5,494,673 titled “TABLES 1-15,” and the subject matters disclosed thereunder are hereby incorporated by reference in their entireties. The instant invention is an improvement on the use of the polyphosphazene polyelectrolytes disclosed in U.S. Pat. No. 5,494,673, and an improvement on the use of other polyphosphazene polyelectrolytes disclosed elsewhere, as immunoadjuvants. The instant invention is also an improvement on the use of the polyphosphazene polyelectrolyte and antigen combinations disclosed in U.S. Pat. No. 5,494,673, and an improvement on the use of other polyphosphazene polyelectrolyte and antigen combinations disclosed elsewhere. The instant invention is also an improvement on the use of the polyphosphazene adjuvants disclosed in U.S. Patent Application Publication 20060193820 (filed Aug. 31, 2006). The disclosures of U.S. Patent Application Publication 20060193820 are hereby incorporated by reference in their entireties.

In accordance with one embodiment of the invention there is provided a product comprising an antigen and a polyphosphazene polyelectrolyte adjuvant each present therein in an amount such that when the product is administered intradermally, or is applied intradermally, to a human or to an animal, the product elicits an immune response in the human or in the animal, against the antigen.

In accordance with one embodiment of the invention there is provided a method for producing an immune response in a human or in an animal comprising producing an immune response in a human or in an animal by intradermally administering to the human or to the animal an antigen and a polyphosphazene polyelectrolyte adjuvant each present in an amount effective to elicit an immune response in the human or in the animal against the antigen.

In accordance with an aspect of some embodiments of the invention, the antigen and the polyphosphazene polyelectrolyte adjuvant are combined together in a liquid form.

In accordance with an aspect of some embodiments of the invention, the antigen and the polyphosphazene polyelectrolyte adjuvant are combined together in a solid form.

In some embodiments of the invention the immune response that is elicited, in the human or in the animal, against the antigen, is a protective (or prophylactic) immune response.

In some embodiments of the invention the immune response that is elicited, in the human or in the animal, against the antigen, is a treating (or therapeutic) immune response.

In some embodiments of the invention the immune response that is elicited, in the human or in the animal, against the antigen, is both a protective (or prophylactic) immune response and a treating (or therapeutic) immune response.

As used hereinabove and hereinbelow any of the terms “intradermal immunization,” “intradermal delivery,” “intradermal vaccination,” “intradermal administration,” “intradermal application,” and “intradermal injection” shall each mean non-parenteral application on to or into the skin.

As used hereinabove and hereinbelow any of the terms “intradermally immunizing,” “intradermally delivering,” “intradermally vaccinating,” “intradermally administering,” “intradermally applying,” and “intradermally injecting” shall each mean non-parenterally applying on to or into the skin.

As used hereinabove and hereinbelow any of the terms “intradermally immunized,” “intradermally delivered,” “intradermally vaccinated,” “intradermally administered,” “intradermally applied,” and “intradermally injected” shall each mean non-parenterally applied on to or into the skin.

As used hereinabove and hereinbelow none of the terms “intradermal immunization,” “intradermal delivery,” “intradermal vaccination,” “intradermal administration,” “intradermal application,” or “intradermal injection” shall ever mean any of intramuscular immunization, intramuscular delivery, intramuscular vaccination, intramuscular administration, intramuscular application, intramuscular injection, subcutaneous immunization, subcutaneous delivery, subcutaneous vaccination, subcutaneous administration, subcutaneous application, or subcutaneous injection.

As used hereinabove and hereinbelow none of the terms “intradermally immunizing,” “intradermally delivering,” “intradermally vaccinating,” “intradermally administering,” “intradermally applying,” or “intradermally injecting” shall ever mean any of intramuscularly immunizing, intramuscularly delivering, intramuscularly vaccinating, intramuscularly administering, intramuscularly applying, intramuscularly injecting, subcutaneously immunizing, subcutaneously delivering, subcutaneously vaccinating, subcutaneously administering, subcutaneously applying, or subcutaneously injecting.

As used hereinabove and hereinbelow none of the terms “intradermally immunized,” “intradermally delivered,” “intradermally vaccinated,” “intradermally administered,” “intradermally applied,” or “intradermally injected” shall ever mean any of intramuscularly immunized, intramuscularly delivered, intramuscularly vaccinated, intramuscularly administered, intramuscularly applied, intramuscularly injected, subcutaneously immunized, subcutaneously delivered, subcutaneously vaccinated, subcutaneously administered, subcutaneously applied, or subcutaneously injected.

The research community and the pharmaceutical industry are striving to develop novel vaccination strategies that can make immunizations painless and safer. Delivering antigens, vaccines, and like active entities into the skin holds promise for achieving these goals.

The skin is made up of several layers with the upper composite layer being the epithelial layer. The outermost layer of the skin is the stratum corneum which has well known barrier properties to prevent molecules and various substances from entering the body and analytes from exiting the body. The stratum corneum is a complex structure of compacted keratinized cell remnants having a thickness of about 10-30 microns. The stratum corneum forms a waterproof membrane to protect the body from invasion by various substances and the outward migration of various compounds.

The natural impermeability of the stratum corneum prevents the administration of most pharmaceutical agents and other substances through the skin. Numerous methods and devices have been proposed to enhance the permeability of the skin and to increase the diffusion of various drugs through the skin so that the drugs can be utilized by the body. Typically, the delivery of drugs through the skin is enhanced by either increasing the permeability of the skin or increasing the force or energy used to direct the drug through the skin.

One example of a method for increasing the delivery of drugs through the skin include iontophoresis. Iontophoresis generally applies an external electrical field to ionize the drug, thereby increasing the diffusion of the drug through the skin.

Sonic, and particularly ultrasonic energy, has also been used to increase the diffusion of drugs through the skin. The sonic energy is typically generated by passing an electrical current through a piezoelectric crystal or other suitable electromechanical device.

Another method of delivering drugs through the skin is by forming micropores or cuts through the stratum corneum. By penetrating the stratum corneum and delivering the drug to the skin in or below the stratum corneum, many drugs can be effectively administered. The devices for penetrating the stratum corneum generally include a plurality of micron size needles or blades having a length to penetrate the stratum corneum without passing completely through the epidermis. Examples of these devices are disclosed in U.S. Pat. No. 5,879,326 to Godshall et al.; U.S. Pat. No. 5,250,023 to Lee et al., and WO 97/48440, the disclosures of each of which are hereby incorporated by reference in their entireties.

Other methods of intradermal delivery of active ingredients are known to use pulsed laser light to ablate the stratum corneum without significant ablation or damage to the underlying epidermis. A drug is then applied to the ablated area and allowed to diffuse through the epidermis.

Other methods of intradermal delivery include those delivery methods that are referred to as being “transcutaneous” in each of U.S. Patent Application Publication 2004/0028727, U.S. Patent Application Publication 2004/0109869, U.S. Patent Application Publication 2005/0287157, and U.S. Patent Application Publication 2007/008248, the disclosures of each of which are hereby incorporated by reference in their entireties. Included among these methods are transcutaneous immunization systems delivering antigen to immune cells through the skin, using a skin-active adjuvant (e.g., an ADP-ribosylating exotoxin) to induce an antigen-specific immune response (e.g., humoral and/or cellular effectors) after transcutaneous application of a dry formulation containing antigen and adjuvant to the skin. The dry formulation may be a powder or a unit-dose patch. Transcutaneous immunization may be induced with or without penetration enhancement. Also included among these methods are devices for the disruption of one or more layers of skin to administer therapeutic agents, e.g., antigens or drugs. The devices are designed to disrupt a defined area of skin. The defined area can approximate the area that a patch or other suitable vehicle for therapeutic agent, e.g., drug or vaccine, delivery is designed to contact. Exemplary devices employ a mask to define the area to be disrupted. Other devices disrupt a defined area by rotating in place. For devices that employ a mask that is secured to the skin, there are employed methods of disrupting the stratum corneum by first securing the mask to the skin and then disrupting the skin. For rotating devices, the disrupting member is simply placed against the skin and actuated to effect disruption.

Still other methods of intradermal delivery of active ingredients take the form of topically applied compositions that include permeation enhancers. Non-limiting examples of permeation enhancers useful in the instant invention are the simple long chain esters that are Generally Recognized As Safe (GRAS) in the various pharmacopoeial compendia. These may include simple aliphatic, unsaturated or saturated (but preferably fully saturated) esters, which contain up to medium length chains. Non-limiting examples of such esters include myristyl myristate, octyl palmitate, and the like. The enhancers are of a type that are suitable for use in a pharmaceutical composition. The enhancer is present in the composition in a concentration effective to enhance penetration of the active ingredient through the stratum corneum and delivering the drug to the skin in or below the stratum corneum. Various considerations should be taken into account in determining the amount of enhancer to use. Such considerations include, for example, the amount of flux (rate of passage through the stratum corneum) achieved and the stability and compatibility of the components in the formulations.

The use of conventional needles and syringes may also be used for effecting the intradermal delivery of vaccines. Below the dermis layer is subcutaneous tissue (also sometimes referred to as the hypodermics layer) and muscle tissue, in that order. Generally, the outer skin layer, epidermis, has a thickness of between 50 to 200 microns, and the dermis, the inner and thicker layer of the skin, has a thickness between 1.5 to 3.5 millimeters. An intradermal injection is effected by delivering the substance into the dermis of the patient. The most common intradermal method of delivery using a conventional needle and syringe is the Mantoux-style injection. The Mantoux technique involves inserting the needle into the skin laterally, then “snaking” the needle further into the intradermal tissue. Typically, the skin is stretched and a needle cannula is inserted into the skin at an angle varying from around 10 to 15 degrees relative to the plane of the skin. Once the cannula is inserted, fluid is injected to form a blister or wheal in the dermis in which the substance is deposited or otherwise contained. The formation of the wheal ensures proper delivery of the substance into the intradermal layer of the skin.

One very promising technology for intradermal delivery is microneedle-based delivery to the skin. Microneedles are sharp sub-millimeter structures that can penetrate the skin non-invasively and painlessly. Microneedles can be used in a variety of ways to deliver vaccines/drugs into the skin. One method is by coating the vaccine/drug onto solid microneedles; the coating then dissolves in the aqueous environment of the skin upon penetration. Another method comprises intradermal delivery of the vaccine/drug from a reservoir through hollow microneedles.

Each of these intradermal vaccination methods may be a more efficacious route of vaccination than intramuscular or subcutaneous vaccination, and could be a potential boon to dealing with future vaccination shortages and mass vaccination crises.

In accordance with one embodiment of the invention there is provided an iontophoresis product comprising an external electric field, and an antigen and a polyphosphazene polyelectrolyte adjuvant each present therein in an amount such that when the product is administered intradermally, or is applied intradermally, to a human or to an animal, the product elicits an immune response in the human or in the animal, against the antigen.

In accordance with one embodiment of the invention there is provided an iontophoresis method for producing an immune response in a human or in an animal comprising producing an immune response in a human or in an animal by intradermally administering to the human or to the animal an antigen and a polyphosphazene polyelectrolyte adjuvant each present in an amount effective to elicit an immune response in the human or in the animal against the antigen, by way of diffusing the antigen and a polyphosphazene polyelectrolyte adjuvant through the skin by the application of an external electric field.

In accordance with one embodiment of the invention there is provided a sonic product comprising an electrical current, a piezoelectric crystal or other suitable electromechanical device, and an antigen and a polyphosphazene polyelectrolyte adjuvant each present therein in an amount such that when the product is administered intradermally, or is applied intradermally, to a human or to an animal, the product elicits an immune response in the human or in the animal, against the antigen.

In accordance with one embodiment of the invention there is provided a sonic method for producing an immune response in a human or in an animal comprising producing an immune response in a human or in an animal by intradermally administering to the human or to the animal an antigen and a polyphosphazene polyelectrolyte adjuvant each present in an amount effective to elicit an immune response in the human or in the animal against the antigen, by way of diffusing the antigen and a polyphosphazene polyelectrolyte adjuvant through the skin by the application of sonic energy generated by passing an electrical current through a piezoelectric crystal or other suitable electromechanical device.

In accordance with one embodiment of the invention there is provided an ultrasonic product comprising an electrical current, a piezoelectric crystal or other suitable electromechanical device, and an antigen and a polyphosphazene polyelectrolyte adjuvant each present therein in an amount such that when the product is administered intradermally, or is applied intradermally, to a human or to an animal, the product elicits an immune response in the human or in the animal, against the antigen.

In accordance with one embodiment of the invention there is provided an ultrasonic method for producing an immune response in a human or in an animal comprising producing an immune response in a human or in an animal by intradermally administering to the human or to the animal an antigen and a polyphosphazene polyelectrolyte adjuvant each present in an amount effective to elicit an immune response in the human or in the animal against the antigen, by way of diffusing the antigen and a polyphosphazene polyelectrolyte adjuvant through the skin by the application of ultrasonic energy generated by passing an electrical current through a piezoelectric crystal or other suitable electromechanical device.

In accordance with one embodiment of the invention there is provided a micropore-forming/cut-forming product comprising a plurality of micron size needles or blades having a length to penetrate the stratum corneum without passing completely through the epidermis, and an antigen and a polyphosphazene polyelectrolyte adjuvant each present therein in an amount such that when the product is administered intradermally, or is applied intradermally, to a human or to an animal, the product elicits an immune response in the human or in the animal, against the antigen.

In accordance with one embodiment of the invention there is provided a micropore forming method for producing an immune response in a human or in an animal comprising producing an immune response in a human or in an animal by intradermally administering to the human or to the animal an antigen and a polyphosphazene polyelectrolyte adjuvant each present in an amount effective to elicit an immune response in the human or in the animal against the antigen, by way of a plurality of micron size needles or blades having a length to penetrate the stratum corneum without passing completely through the epidermis.

In accordance with one embodiment of the invention there is provided an ablation product comprising pulsed laser light, and an antigen and a polyphosphazene polyelectrolyte adjuvant each present therein in an amount such that when the product is administered intradermally, or is applied intradermally, to a human or to an animal, the product elicits an immune response in the human or in the animal, against the antigen.

In accordance with one embodiment of the invention there is provided an ablation method for producing an immune response in a human or in an animal comprising producing an immune response in a human or in an animal by intradermally administering to the human or to the animal an antigen and a polyphosphazene polyelectrolyte adjuvant each present in an amount effective to elicit an immune response in the human or in the animal against the antigen, by way of using pulsed laser light to ablate the stratum corneum without significantly ablating or damaging the underlying epidermis, then applying the antigen and polyphosphazene polyelectrolyte adjuvant to the ablated area and allowing them to diffuse through the epidermis.

In accordance with one embodiment of the invention there is provided a topically applied composition product comprising a permeation enhancer, and an antigen and a polyphosphazene polyelectrolyte adjuvant each present therein in an amount such that when the product is administered intradermally, or is applied intradermally, to a human or to an animal, the product elicits an immune response in the human or in the animal, against the antigen.

In accordance with one embodiment of the invention there is provided a topically applied method for producing an immune response in a human or in an animal comprising producing an immune response in a human or in an animal by intradermally administering to the human or to the animal a permeation enhancer, and an antigen and a polyphosphazene polyelectrolyte adjuvant each present in an amount effective to elicit an immune response in the human or in the animal against the antigen.

In accordance with one embodiment of the invention there is provided a conventional needle and syringe product comprising a conventional needle and syringe, and an antigen and a polyphosphazene polyelectrolyte adjuvant each present therein in an amount such that when the product is administered intradermally, or is applied intradermally, to a human or to an animal, the product elicits an immune response in the human or in the animal, against the antigen.

In accordance with one embodiment of the invention there is provided a conventional needle and syringe method for producing an immune response in a human or in an animal comprising producing an immune response in a human or in an animal by intradermally administering to the human or to the animal an antigen and a polyphosphazene polyelectrolyte adjuvant each present in an amount effective to elicit an immune response in the human or in the animal against the antigen, by way of intradermally injecting the antigen and the polyphosphazene polyelectrolyte adjuvant into the skin of the human or the animal.

In accordance with one embodiment of the invention there is provided a solid microneedle product comprising a microneedle, and an antigen and a polyphosphazene polyelectrolyte adjuvant each present therein in an amount such that when the product is administered intradermally, or is applied intradermally, to a human or to an animal, the product elicits an immune response in the human or in the animal, against the antigen.

Solid microneedles can contain adjuvant and antigen in the form of a coating, or the entire microneedle can be fabricated using antigen and adjuvant (dissolvable polymer microneedles).

In accordance with one embodiment of the invention there is provided a solid microneedle method for producing an immune response in a human or in an animal comprising producing an immune response in a human or in an animal by intradermally administering to the human or to the animal an antigen and a polyphosphazene polyelectrolyte adjuvant each present in an amount effective to elicit an immune response in the human or in the animal against the antigen, by way of a microneedle upon which the antigen and the polyphosphazene polyelectrolyte adjuvant are coated.

In accordance with one embodiment of the invention there is provided a solid microneedle method for producing an immune response in a human or in an animal comprising producing an immune response in a human or in an animal by intradermally administering to the human or to the animal an antigen and a polyphosphazene polyelectrolyte adjuvant each present in an amount effective to elicit an immune response in the human or in the animal against the antigen, by way of an entire microneedle microfabricated using the antigen and the polyphosphazene polyelectrolyte adjuvant.

In accordance with one embodiment of the invention there is provided a hollow microneedle product comprising a microneedle, and an antigen and a polyphosphazene polyelectrolyte adjuvant each present therein in an amount such that when the product is administered intradermally, or is applied intradermally, to a human or to an animal, the product elicits an immune response in the human or in the animal, against the antigen.

In accordance with one embodiment of the invention there is provided a hollow microneedle method for producing an immune response in a human or in an animal comprising producing an immune response in a human or in an animal by intradermally administering to the human or to the animal an antigen and a polyphosphazene polyelectrolyte adjuvant each present in an amount effective to elicit an immune response in the human or in the animal against the antigen, by way of a microneedle through which the antigen and the polyphosphazene polyelectrolyte adjuvant are communicated from a reservoir and into the skin.

In a preferred embodiment the invention applies the antigen and adjuvant as a coating for asperities or microneedles. More particularly in one preferred embodiment of this invention the coating formulation, includes at least one biologically active agent and at least one polyphosphazene polyelectrolyte, and further relates to asperities or microprojections or microneedles coated with such formulations.

As the above discussion conveys, there is an interest in sequestering, entrapping, encapsulating, and/or depositing various compounds or substances on the surfaces of and/or within various structures, such as, for example, polymer, metal, or ceramic structures. Thus, such structures may be used for the topical delivery of biologically active agents. Topical delivery of biologically active agents is a useful method for achieving systemic or localized pharmacological effects. For example, U.S. Pat. No. 3,964,482, issued to Gerstel, discloses an array of either solid or hollow microneedles for penetrating through the stratum corneum, into the epidermal layer; that patent is incorporated herein by reference in its entirety.

Methods for coating of microneedles to form a solid drug containing formulations have been described previously. U.S. Pat. No. 6,855,372 describes a method of coating a liquid on microprojections without coating the liquid on the substrate using a roller, and immersing microprojections to a predetermined level, and is incorporated herein by reference in its entirety. Gill, H. S. et al., Journal of Controlled Release, 117 (2007) 227-237, describes a process for fabricating the coating on microneedles via micro dip-coating in a reservoir containing a cover to restrict the access of liquid only to the microneedle shaft, and is incorporated herein by reference in its entirety. Both of these methods rely on varying the number of contacts (dips) between the microneedle and the reservoir or roller to control a dosage of biologically active compound to be coated on the microneedle.

PCT Application No. PCT/US06/23814 also describes methods for coating of microneedles to form solid drug containing formulations by multiple contacts between the microneedle and the coating liquid, and is incorporated herein by reference in its entirety.

It is an object of the present invention to provide a coating for asperities or microneedles for intradermal delivery of a biologically active agent, which provides for improved loading of the biologically active agent on the asperities or microneedles.

In accordance with an aspect of the present invention there is provided a formulation for coating asperities. The formulation comprises at least one antigen and at least one polyphosphazene polyelectrolyte.

The term “asperities,” as used herein, means the microscopic surface elevations present on the surface of a material, such as pins, microprojections, and microneedles.

The asperities, microprojections, and microneedles preferably are in the form of piercing elements which are dimensioned to penetrate into the skin or which may deliver a biological material intradermally. In a non-limiting embodiment, the asperity, microprojection, or microneedle is dimensioned such that it penetrates through the stratum corneum into the underlying epidermis layer, and in some embodiments, the dermal layer of the skin.

In a non-limiting embodiment, the polyphosphazene polyelectrolyte is at least partially soluble in water (typically to an extent of at least 0.001 wt. %), an aqueous buffered salt solution, or an aqueous alcohol solution. The polyphosphazene polyelectrolyte, in a non-limiting embodiment, contains charged side groups, either in the form of an acid or base that is in equilibrium with its counterion, or in the form of an ionic salt thereof.

In a non-limiting embodiment, the polyphosphazene polyelectrolyte is biodegradable and exhibits minimal toxicity when administered to animals, including humans.

Polyphosphazenes are polymers with backbones consisting of alternating phosphorus and nitrogen, separated by alternating single and double bonds. Each phosphorous atom is covalently bonded to two pendant groups (“R”). The repeat unit in polyphosphazenes has the following general formula:

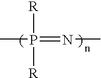

wherein n is an integer. Each R may be the same or different.

In a non-limiting embodiment, the polyphosphazene has only one type of pendant group or side group repeatedly attached to its backbone, and the polymer is a homopolymer. In another non-limiting embodiment, the polyphosphazene has more than one type of pendant group and the groups vary randomly or regularly throughout the polymer. The phosphorus thus can be bound to two like groups, or to two different groups.

In a non-limiting embodiment, the polymers of the present invention may be produced by producing initially a reactive macromolecular precursor such as, but not limited to, poly(dichlorophosphazene). The pendant groups then are substituted onto the polymer backbone by reaction between the reactive chlorine atoms on the backbone and the appropriate organic nucleophiles, such as, for example, alcohols, amines, or thiols. Polyphosphazenes with two or more types of pendant groups can be produced by reacting a reactive macromolecular precursor such as poly(dichlorophosphazene) with two or more types of nucleophiles in a desired ratio. Nucleophiles can be added to the reaction mixture simultaneously or in sequential order. The resulting ratio of pendant groups in the polyphosphazene will be determined by a number of factors, including the ratio of starting materials used to produce the polymer, the order of addition, the temperature at which the nucleophilic substitution reaction is carried out, and the solvent system used. While it is difficult to determine the exact substitution pattern of the groups in the resulting polymer, the ratio of groups in the polymer can be determined easily by one skilled in the art.

Polyphosphazene polyelectrolytes useful in the present invention are, in a non-limiting embodiment, polyphosphazenes containing ionic or charged moieties in their pendant groups, such as carboxylic acid, sulfonic acid, and amino groups, which can be in the acidic, basic, or salt forms. Examples of such groups include -phenylCO₂H, -phenylSO₃H, -phenylPO₃H, -(aliphatic)CO₂H, -(aliphatic)SO₃H, -(aliphatic)PO₃H, -phenyl(aliphatic)CO₂H, -phenyl(aliphatic)SO₃H, -phenyl(aliphatic)PO₃H, —[(CH₂)ₓO]_{y}phenylCO₂H, —[(CH₂)ₓO]_{y}phenylSO₃H, —[(CH₂)ₓO]_{y}phenylPO₃H, —[(CH₂)ₓO]_{y}(aliphatic)CO₂H, —[(CH₂)ₓO]_{y}(aliphatic)SO₃H, —[(CH₂)ₓO]_{y}(aliphatic)PO₃H, —[(CH₂)ₓO]_{y}phenyl(aliphatic)CO₂H, —[(CH₂)ₓO]_{y}phenyl(aliphatic)SO₃H, —[(CH₂)ₓO]_{y}phenyl(aliphatic)PO₃H, -alkylamines, -arylamines, -alkylarylamines, -arylalkylamines, —[(CH₂)ₓO]_{y}alkylamines, —[(CH₂)ₓO]_{y}arylamines, —[(CH₂)ₓO]_{y}alkylarylamines, —[(CH₂)ₓO]_{y}arylalkylamines, wherein x is 1-8 and y is an integer of 1 to 20. The amines, when present, may be primary, secondary, tertiary, or quaternary. The groups can be bonded to the phosphorous atom through, for example, an oxygen, sulfur, nitrogen, or carbon atom.

The polyphosphazenes of the present invention can be homopolymers, having one type of side groups, or mixed substituent copolymers, having two or more types of side groups. When polyphosphazene polymers of the present invention are copolymers and have two or more different types of side groups they can contain either different types of ionic groups or a combination of ionic and non-ionic groups. Side groups that do not contain ionic functionalities can be introduced in a polyphosphazene copolymer to modulate physical or physico-chemical properties of the polymer. Such side groups can be used, for example, to improve water solubility, to modulate biodegradability, to increase hydrophobicity, or to change chain flexibility of the polymer. These side groups (other than ionic groups as described above) may be one or more of a wide variety of substituent groups. As representative, non-limiting examples of such groups there may be mentioned: aliphatic; aryl; aralkyl; alkaryl; heteroaromatic; carbohydrates, including glucose, mannose; heteroalkyl; halogen; -oxyaryl including but not limited to -oxyphenyl, -oxyphenylhydroxyl; -oxyaliphatic including -oxyalkyl, and -oxy(aliphatic)hydroxyl, including oxy(alkyl)hydroxyl; -oxyalkaryl, -oxyaralkyl; -thioaryl; thioaliphatic including -thioalkyl; -thioalkaryl; thioaralkyl; aminoalkyl, aminoaryl, N-Ethylpyrrolidone, such as 2-(2-oxo-1-pyrrolidinyl)ethoxy; —NH—[(CH₂)ₓ—O-]_{y}-(aryl or aliphatic); and —O—[(CH₂)ₓ—O-]_{y}-(aryl or aliphatic); wherein x is 1-8 and y is an integer of 1 to 20.

In a non-limiting embodiment, the polymers of the present invention are homopolymers containing carboxylic acid side groups, such as poly[di(carboxylatophenoxy)phosphazene], or PCPP, and poly[di(carboxylatophenoxyethyl)phosphazene], and salts thereof, such as sodium salts, for example.

In a non-limiting embodiment, the polyphosphazene polyelectrolytes, such as one containing carboxylic acid groups can be produced as follows. An organic compound containing hydroxyl group and ester group may be reacted with reactive chlorine atoms on the polymer backbone. One or a mixture of organic compounds can be used to result in a homopolymer or a copolymer having more than one type of pendant group. Hydroxyl groups of the organic compound can be activated with sodium, sodium hydride, or sodium hydroxide by procedures known in the art and then reacted with chlorine atoms attached to the polyphosphazene backbone. After the completion of the reaction, the ester functionalities of the pendant groups may be hydrolyzed to yield carboxylic acid functionalities. All ester functionalities can be hydrolyzed to achieve full conversion into the acid groups, or, if desired, the reaction can be stopped before completion, thereby resulting in a substituted copolymer containing both acid and ester functionalities. The polymer then can be dissolved in an aqueous solution at a desired concentration. The acid groups also can be converted into salt form, such as sodium or potassium, if required to improve solubility or to achieve desired polymer conformation and physicochemical characteristics.

In a non-limiting embodiment, the polyphosphazene polymer has an overall molecular weight of 5,000 g/mol to 10,000,000 g/mol, and in another embodiment from 40,000 g/mol to 1,000,000 g/mol.

The polyphosphazenes of the present invention, in a non-limiting embodiment, are polymers that may be biodegradable when administered to either humans or animals. Biodegradability of the polymer prevents eventual deposition and accumulation of polymer molecules at distant sites in the body, such as the spleen. The term biodegradable, as used herein, means a polymer that degrades within a period that is acceptable in the desired application, typically less than about five years and most preferably less than about one year.

The polyphosphazenes may be cross-linked ionically after being coated on an asperity, microprojection, or microneedle. ionically cross-linkable polyphosphazenes, for example, can be cross-linked by treating a phosphazene polymer with a multivalent metal cation such as zinc, calcium, bismuth, barium, magnesium, aluminum, copper, cobalt, nickel, cadmium, or other multivalent metal cation known in the art; or with a multivalent organic cation such as spermine, spermidine, poly(ethyleneimine), poly(vinylamine), or other multivalent organic cation known in the art. Ionic cross-linking of the coating may be desired to improve the mechanical strength of the coating or to modulate the release of the at least one biologically active agent.

The liquid formulation of the present invention comprises any liquid that is compatible with the polyphosphazene of the present invention and the biologically active compound. It can be a solution or a dispersion, such as an emulsion or suspension. It can be water based or can contain organic solvents, or a mixture of water and organic solvents. In one embodiment, the formulation is a water or an aqueous based formulation. It can contain salts, acids, bases, or other excipients to maintained a desired pH and ionic strength.

In a non-limiting embodiment, the at least one polyphosphazene polyelectrolyte is present in the formulation in an amount of from about 0.0001% (wt./vol.) to about 30% (wt./vol.). In another non-limiting embodiment, the at least one polyphosphazene polyelectrolyte is present in the formulation in an amount of from about 0.01% (wt./vol.) to about 5% (wt./vol.)

Biologically active agents which may be included in the formulation are vaccine antigens. The vaccine antigens of the invention can be derived from a cell, a bacterium or virus particle or a portion thereof. The antigen can be a protein, peptide, polysaccharide, glycoprotein, glycolipid, or combination thereof which elicits an immunogenic response in a human; or in an animal, for example, a mammal, bird, or fish. The immunogenic response can be humoral, mucosal, or cell mediated. Examples are viral proteins, such as influenza proteins, human immunodeficiency virus (HIV) proteins, Herpes virus proteins, and hepatitis A and B proteins. Additional examples include antigens derived from rotavirus, measles, mumps, rubella, and polio; or from bacterial proteins and lipopolysaccharides such as Gram-negative bacterial cell walls. Further antigens may also be those derived from organisms such as Haemophilus influenza, Clostridium antigens, including but not limited to, Clostridium tetani, Corynebacterium diphtheria, and Nesisseria gonhorrhoae, as well as anthrax antigens. Additional examples include those found in U.S. Patent Application Publication 20070292386, the disclosures of which are hereby incorporated by reference in their entireties. Those found at ¶ [0051] and [0052] U.S. Patent Application Publication 20070292386 are more specifically incorporated by reference in their entireties.

In a non-limiting embodiment, the at least one biologically active agent is present in the formulation in an amount effective to provide a desired biological effect or result. In a non-limiting embodiment, the at least one biologically active agent is present in the formulation in an amount of from about 0.0001% (wt./vol.) to about 70% (wt./vol.). In another non-limiting embodiment, the at least one biologically active agent is present in the formulation in an amount of from about 0.005% (wt./vol.) to about 10% (wt./vol.)

In a non-limiting embodiment, the liquid formulation also may include vaccine adjuvants or immunostimulating compounds which, when the at least one biologically active agent is an antigen, enhance an immune response to the antigen in the recipient host. The liquid formulation may also include immune response modifying compounds, compounds that act through basic immune system mechanisms known as toll like receptors to induce selected cytokine biosynthesis. Typical examples of adjuvants and immune modulating compounds include, but are not limited to, aluminum hydroxide, aluminum phosphate, squalene, Freunds adjuvant, certain poly- or oligonucleotides (DNA sequences), such as CpG, Ribi adjuvant system, polyphosphazene adjuvants such as poly[di(carboxylatophenoxy)phosphazene] (PCPP) and poly[di(carboxylatoethylphenoxy) phosphazene] (PEPP), MF-59, saponins, such as saponins purified from the bark of the Q. saponaria tree, such as QS-21, derivatives of lipopolysaccharides, such as monophosphoryl lipid (MPL), muramyl dipeptide (MDP) and threonyl muramyl dipeptide (tMDP); OM-174; non-ionic block copolymers that form micelles such as CRL 1005; and Syntex Adjuvant Formulation. In case of polyphosphazene immunostimulating compounds, the compounds can act as both the adjuvants and the additives for the liquid formulation. The liquid coating fluid formulation also may include one or more pharmaceutical acceptable and/or approved additives (excipients), antibiotics, preservatives, diluents and stabilizers. Such substances include but are not limited to water, saline, glycerol, ethanol, wetting or emulsifying compounds, pH buffering substances, stabilizing compounds such as polyols, for example trehalose, or the like.

In a non-limiting embodiment, the at least one biologically active agent may be formulated or encapsulated in various forms or encapsulation media, such as in microspheres, nanospheres, microcapsules, nanocapsules, microgels, nanogels, liposomes, or dendrimers. The above-mentioned forms may modulate the release profile in order to achieve a desirable biological (therapeutic) effect. For example, such forms may provide a controlled release of at least one biologically active agent over a desired period of time.

In another non-limiting embodiment, the formulation further comprises at least one surface tension reducing agent.

In a non-limiting embodiment, the at least one surface tension reducing agent is at least one surfactant. In yet another non-limiting embodiment, the at least one surfactant may be an anionic surfactant, a cationic surfactant, or a non-ionic surfactant.

Anionic surfactants which may be employed include sulfates such as alkyl sulfates (for example, sodium dodecyl sulfate), ammoniumlauryl sulfate, sodium lauryl ether sulfate, sulfated fats and oils, sulfated oleic acid, sulfated alkanolamides, sulfated esters, and alcohol sulfates; sulfonates such as alkylaryl sulfonates, olefin sulfonates, ethoxylated alcohol sulfates, and sulfonates of ethoxylated alkyl phenols; sulfates of fatty esters; sulfates and sulfonates of alkyl phenols; lignosulfonates; sulfonates of condensed naphthalenes; sulfonates of naphthalene; dialkyl sulfosuccinates, including sodium derivatives; sodium derivatives of sulfosuccinates, such as the disodium ethoxylated nonyl phenol half ester of sulfosuccinic acid, the disodium ethoxylated alcohol (C₁₀-C₁₁), half-ester of sulfosuccinic acids, etc., petroleum sulfonates, such as alkali salts of petroleum sulfonates; for example, sodium petroleum sulfonate (Acto 632); phosphate esters, such as alkali phosphate esters, and a potassium salt of phosphate ester (Triton H66); sulfonated alkyl esters (for example, Triton GR 7); carboxylates, such as those of the formula (RCOO)−(M)+ wherein R is an alkyl group having from 9-21 carbon atoms, and M is a metal or an amine; and sodium polymeric carboxylic acid (Tamol 731) and the like.

In one non-limiting embodiment, the anionic surfactant is selected from the group consisting of sodium dodecyl sulfate, ammoniumlauryl sulfate, and sodium lauryl ether sulfate.

Cationic surfactants which may be employed include quaternary amino or nitrogen compounds; quaternary ammonium salts such as benzalkonium chloride, benzethonium, alkyl-trimethylammonium salts, and alkylpyridinium salts; aliphatic mono-,di-, and polyamines; rosin-derived amines; amine oxides, such as polyoxyethylene alkyl and alicyclic amines, N,N,N,N tetrakis-substituted ethylene diamines, amide-linked amines, such as those prepared by the condensation of a carboxylic acid with a di- or polyamine, and sodium tauro-24, 25-dihydrofusidate.

In a non-limiting embodiment, the cationic surfactant is selected from the group consisting of benzalkonium chloride and benzethonium.

Nonionic surfactants which may be employed include polyoxyethylenes; alkyl polyethylene oxide ethoxylated alkyl phenols, ethoxylated aliphatic alcohols; carboxylic acid esters, such as glycerol esters, polyethylene glycol esters, and polyoxyethylene fatty acid esters; polyoxyethylene sorbitan fatty esters; polyoxyethylene derivatives of fatty acid partial esters of sorbitol anhydrides, including but not limited to, Tween 80, Tween 20, Pluronics, Polyoxynol 40 Stearate, Polyoxyethylene 50 Stearate, and octoxynol; anhydrosorbitol esters and ethoxylated anhydrosorbitol esters; glycol esters of fatty acids; ethoxylated natural fats, oils, and waxes; carboxylic amides, such as diethanolamine condensates, and monoalkanolamine condensates; polyoxyethylene fatty acid amides; polyalkylene oxide block copolymers, such as polyethylene and polypropylene oxide block copolymers; and polysiloxane-polyoxyalkylene copolymers; 1-dodecylazacycloheptan-2-one (Nelson R & D); alkylpolyglucosides; polyethylene glycol monolaurate (Alza); and Macrochem's SEPA nonionic surfactant.

In a non-limiting embodiment, the non-ionic surfactant is selected from the group consisting of alkylpolyethylene oxide, copolymers of polyethylene oxide and polypropylene oxide, alkylpolyglucosides, and polyoxyethylene sorbitan fatty esters, and polyoxyethylene derivatives of fatty acid partial esters of sorbitol anhydrides.

In a non-limiting embodiment, the at least one surface tension reducing agent is present in the formulation in an amount of from about 0.0001% (wt./vol.) to about 10% (wt./vol.). In another non-limiting embodiment, the at least one surface tension reducing agent is present in the formulation in an amount of from about 0.01% (wt./vol.) to about 10% (wt./vol.). In another non-limiting embodiment, the at least one surface tension reducing agent is present in the formulation in an amount of from about 0.1% (wt./vol.) to about 3% (wt./vol.).

In another non-limiting embodiment, the formulation further comprises at least one viscosity enhancing agent. In one non-limiting embodiment, the viscosity enhancing agent may be a polymer, or, in another non-limiting embodiment, may be a sugar such as, for example, sucrose. The polymer may be synthetic, semi-synthetic, or of natural origin. The polymer may be linear, branched, brush- or comb-like, or may be a random, alternate, block, or graft copolymer.

In a non-limiting embodiment, the polymer is a water-soluble polymer. Typical examples of such polymers include, but are not limited to, dextran, polyvinylpyrrolidone, poly(vinyl alcohol), poly(ethylene glycol), poly(ethylene oxide), polyoxymethylene, poly(hydroxyethyl methacrylate), dextran, sodium carboxymethylcellulose, hydroxyethylcellulose, hydroxypropylcellulose, alginic acid, chitosan, poly(glutamic acid), hyaluronic acid, poly(isobutylacrylamide), poly(ethylenimine), polyphosphazenes, especially those that comprise pyrrolidone, ethylene oxide, and carboxylic acid containing side-groups, and copolymers thereof. In a non-limiting embodiment, the polymers either are biodegradable or of sufficiently low molecular weight to be removed from the body through renal clearance.

In yet another non-limiting embodiment, the polymers can be hydrophobic, and in one non-limiting embodiment are biodegradable hydrophobic polymers. Examples of hydrophobic polymers are poly(hydroxyvalerate), poly(lactide), poly(glycolide), polycaprolactone, poly(lactide-co-glycolide), poly(hydroxybutyrate), poly(hydroxybutyrate-co-valerate), polydioxanone, polyorthoester, polyanhydride, poly(vinyl methyl ether), polyvinylidene chloride, poly(butyl methacrylate), poly(ethylmethacrylate), poly(vinylidene fluoride), poly(trimethylene carbonate), poly(iminocarbonate), and other derivatized polyurethanes, polyphosphazenes, such as polyaminophosphazenes, including those with amino acid and imidazol side groups, and poly(organosiloxanes). When the polymer is a hydrophobic polymer, the formulation, in a non-limiting embodiment, may further include organic solvents or other compounds that improve the compatibility of such hydrophobic polymers with the polyphosphazene polyelectrolyte and the at least one biologically active agent.

In a non-limiting embodiment, the at least one viscosity enhancing agent is present in the formulation in an amount of about 70% (wt./vol.). In another non-limiting embodiment, the viscosity enhancing agent is present in the formulation in an amount of from about 0.005% (wt./vol.) to about 10% (wt./vol.).

In a further non-limiting embodiment, the formulation further includes one or more pharmaceutically acceptable additives or excipients, such as preservatives, diluents, and stabilizers, such as, for example, saline, glycerol, ethanol, wetting or emulsifying agents, pH buffers, and polyols, such as trehalose.

Preservatives which may be employed include, but are not limited to, benzyl alcohol, parabens, thimerosal, chlorobutanol, and benzalkonium chloride. In a non-limiting embodiment, the preservative is present in the formulation in an amount of from about 0.02% (wt./vol.) to about 2% (wt./vol.)

The at least one polyphosphazene polyelectrolyte polymer and the at least one biologically active agent, and, if desired, the at least one surface tension reducing agent and/or viscosity enhancing agent and/or preservative, are combined in amounts such as those hereinabove described to provide a formulation suitable for coating a substrate which includes a plurality of asperities, microprojections, or microneedles. In a non-limiting embodiment, the formulation is a liquid formulation.

The liquid formulation may be either homogeneous or heterogeneous, such as in the form of a solution, emulsion, or dispersion. The polyphosphazene polyelectrolytes may be combined, in one non-limiting embodiment, with the at least one biologically active agent by mixing a solution (or emulsion or dispersion) of the polyphosphazene polyelectrolyte with a solution of the at least one biologically active agent, and another non-limiting embodiment, either the polyphosphazene polyelectrolyte or the at least one biologically active agent is dissolved or dispersed in a solution, or dispersion that contains the other component.

Physical or physicochemical means can be applied to facilitate the formation of the formulation, such as agitation (stirring, vortexing, shaking), heating, ultrasonic or microwave treatment. It is also understood that the formation of macromolecular complexes between the polyphosphazene polyelectrolyte and the at least one biologically active agent can take place in the formulation. Such macromolecular (or interpolymer, or polyelectrolyte) complexes can be either soluble or insoluble and are produced through the formulation of the non-covalent, such as ionic, hydrogen bonds and/or hydrophobic interactions. Other additives or excipients, such as a surfactant, can contribute to the formation of such complexes. It is also understood that the physical state of the formulation can be different than the physical state of the components. For example, combining a polyphosphazene polyelectrolyte with a dispersion of water-insoluble biologically active agent may result in the formation of a homogeneous water-soluble formulation.

The formulation then is applied to a device including at least one asperity, microprojection, or microneedle to provide a device for delivering at least one biologically active agent which includes at least one asperity, microprojection, or microneedle coated with the formulation.

The formulation can be applied to the asperities, such as microprojections or microneedles, by various methods, such as dip-coating, spin coating, spray coating, electrospinning, electrospraying, or multilayer polyelectrolyte deposition. Dip-coating can be performed using various types of reservoirs suitable for coating the asperities, such as microwells, rollers, hydrogels, or membranes. The coating can be dried to remove the residual solvent, such as water or may be used without drying. If drying is desirable, the coating can be air-dried, or subjected to heat, vacuum, or microwave treatment to facilitate the drying process. Additional steps can be also introduced, such as treatment with volatile solvents to form an azeotrope mixture with a lower boiling point, to accelerate the drying process.

In a non-limiting embodiment, at least one asperity, or microprojection or microneedle is coated with a liquid formulation of the present invention, which includes the at least one polyphosphazene polyelectrolyte and the at least one biologically active agent.

In one non-limiting embodiment, the liquid formulation including the at least one polyphosphazene polyelectrolyte and the at least one biologically active agent is fed from at least one supply reservoir to at least one coating reservoir in an amount sufficient to form at least one layer of a coating on the at least one asperity, microprojection, or microneedle, to provide no more than a predetermined dose of the at least one biologically active agent for the at least one asperity, or microprojection, or microneedle. The at least one asperity, or microprojection, or microneedle is contacted with the liquid formulation to form at least one layer of coating on the at least one asperity, or microprojection, or microneedle. As needed, coating of the at least one asperity, or microprojection, or microneedle with the liquid formulation may be repeated in order to consume substantially the entire amount of the liquid formulation fed to the coating reservoir.

The invention now will be described with respect to the drawings, wherein:

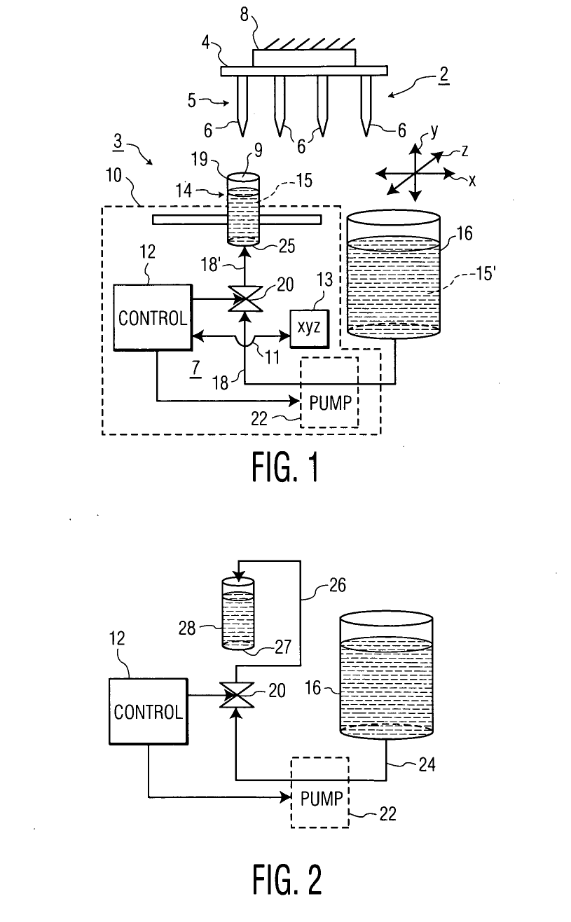

FIG. 1 is a diagrammatic view of a system for coating a microneedle array according to one embodiment of the present invention;

FIG. 2 is a diagrammatic view of a system for coating a microneedle array according to a second embodiment of the present invention;

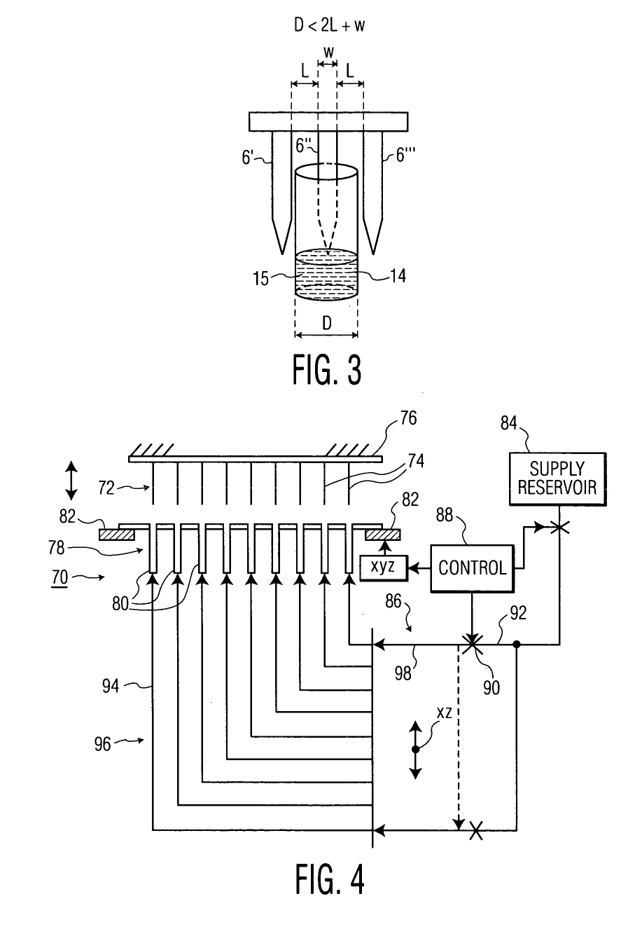

FIG. 3 is a diagrammatic elevational view illustrating certain principles for constructing the systems of FIGS. 1 and 2 according to an embodiment of the present invention;

FIG. 4 is a diagrammatic elevational view of system for coating a microneedle array according to a further embodiment of the present invention;

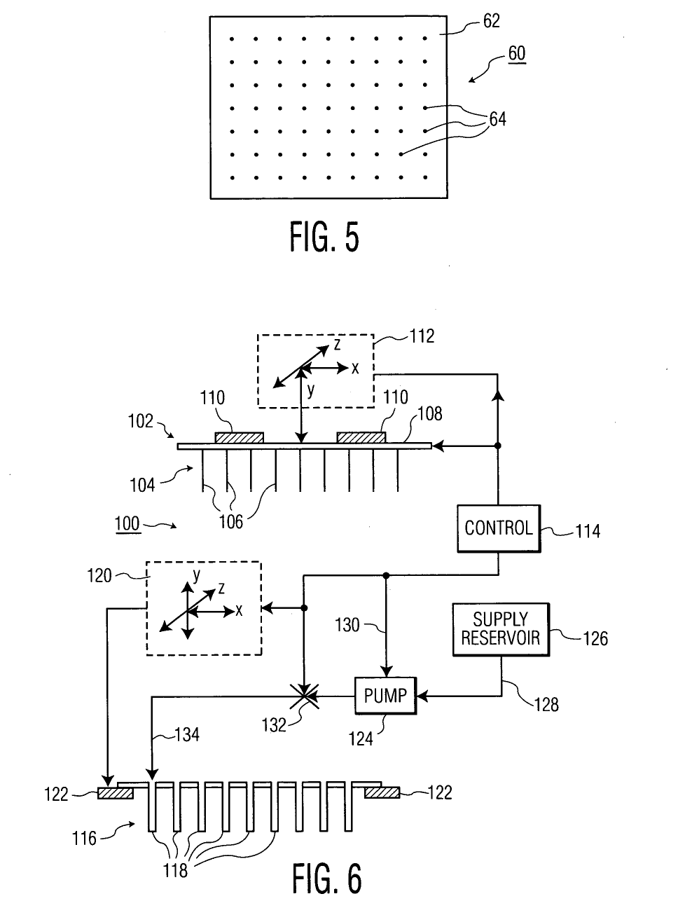

FIG. 5 is a plan view of microneedle array according to a further embodiment of the present invention;

FIG. 6 is a diagrammatic elevational view of system for coating a microneedle array according to a still further embodiment of the present invention;

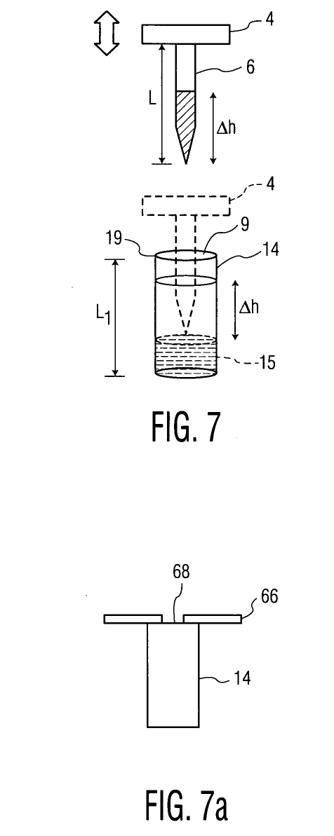

FIG. 7 is an elevational schematic view of a microneedle and its associated coating reservoir according to one embodiment of the present invention;

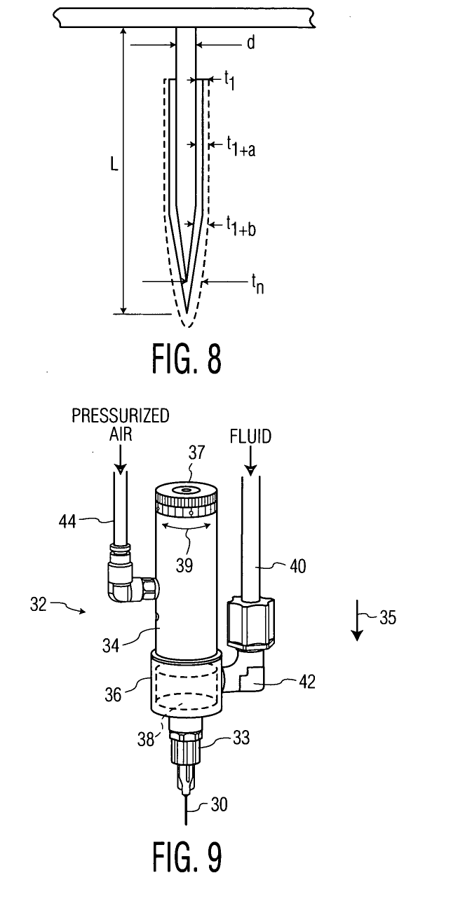

FIG. 8 is an elevational view of a microneedle useful for explaining certain principles of the present invention;

FIG. 9 is a perspective view of a syringe forming an embodiment of a coating reservoir according to one embodiment of the present invention;

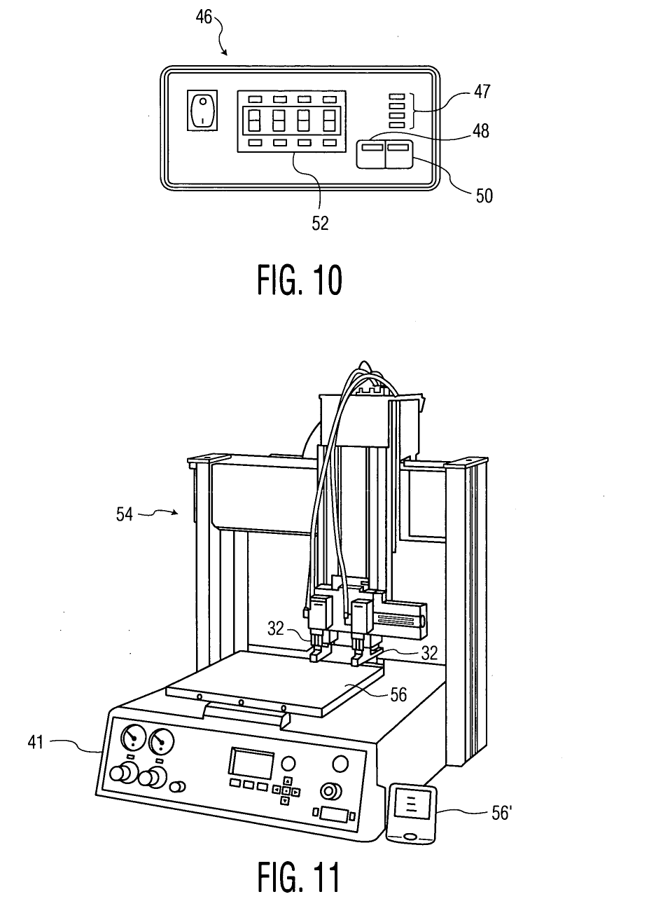

FIG. 10 is a front elevational view of a panel for use on a control apparatus for operating the syringe of FIG. 9;

FIG. 11 is a perspective view of a commercial apparatus employing the syringe of FIG. 9; and

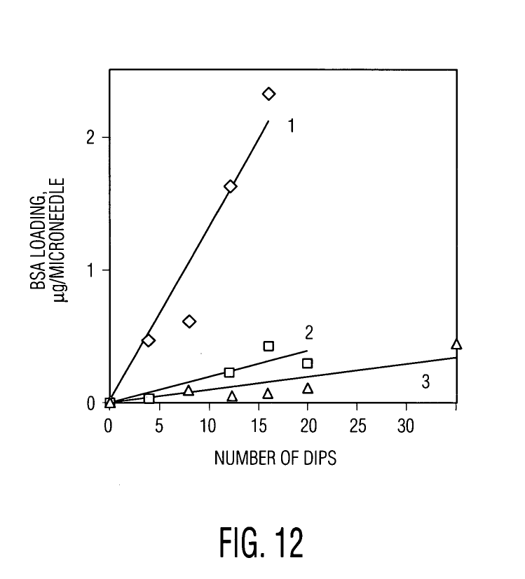

FIG. 12 shows BSA loading per microneedle versus the number of dips for three coating formulations containing 5% (wt./vol.) of BSA in 0.1×PBS solution, and 0.5% (wt./vol.) of PCPP (1); 0.8 (wt./vol.) of CMC (2), and 1.5% (wt./vol) of CMC.

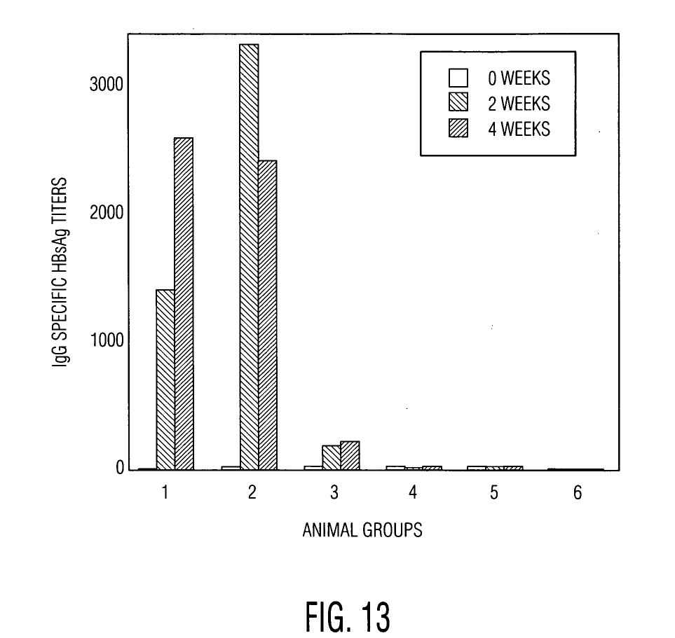

FIG. 13 represents serum IgG specific HBsAg titers after immunization of pigs via intradermal immunization with microneedles with approximately 20 μg of HBsAg formulated with PCPP (Group 1), 10 μg of HBsAg formulated with PCPP (Group 2), and 20 μg of HBsAg (Group 4). Intramuscular immunization with 20 μg of HBsAg formulated with PCPP (Group 3), with 20 μg of HBsAg (Group 6), as well as intradermal injection of 20 μg of HBsAg (Group 5) were used as controls (7 pigs per group; single dose immunization; 0, 2, and 4 week data).

In a non-limiting embodiment there is provided, as shown in FIG. 1, a liquid needle coating system 3 comprises a microneedle array assembly 2 and a coating liquid dispensing system 10. The assembly 2 comprises an array of microneedles 6 attached to a substrate 4. The substrate 4 may be of any suitable material. The dispensing system 10 coats the microneedles 6 with a coating that comprises the liquid formulation comprising a polyphosphazene polyelectrolyte and at least one biologically active compound such as a drug or the like.

The array 5 of microneedles 6 first are coated with a liquid formulation of the present invention comprising at least one polyphosphazene polyelectrolyte and at least one biologically active agent by the system 10. The liquid formulation then is dried to form a final hardened coated set of microneedles 6. The array of microneedles are attached to the skin of a recipient for penetration of the skin by the microneedles in a known manner to deliver the at least one biologically active compound to the recipient through the skin of the recipient and such devices may be referred to as intradermal patches, for example. The coatings disperse the biologically active compound into the flesh of the recipient to administer the biologically active compound. Such microneedles and their coatings are generally known in the art.

The microneedles 6 depend from the substrate 4, which together form the intradermal drug patch or the like for transferring a drug or biologically active agent in a coating applied to the needles 6. The substrate 4 is releasably secured to a support 8 which is fixed in position in this embodiment. In an alternative embodiment, the needles via their support 8 may be positioned by an manifesting no more than the predetermined dose positioning system.

In FIG. 1, dispensing system 10 includes an xyz positioning system 13 coupled to control 10 via bus 11 and a coating fluid dispensing arrangement 7 also under the control of control 10. The system 10 includes a coating fluid reservoir 14 comprising a liquid formulation 15 in a receptacle 19. Container 19 receives the needle coating liquid formulation 15 from a supply reservoir 16 which stores liquid formulation 15′ supplied to reservoir 14 via conduits 18, 18′ through coating fluid metering valve 20. The valve 20 is controlled by control 12. The reservoir 14 contains a liquid formulation including at least one polyphosphazene polyelectrolyte and at least one biologically active agent. The amount of fluid formulation 15 in the reservoir is metered by control 12. Control 12 is a programmed computer that contains instructions for operating the system 10.

The amount of fluid formulation metered to the reservoir 14 in one embodiment is exactly the amount needed to coat one microneedle 6 a predetermined dosage amount of the biological compound that will form the final needle 6 dry dosage coating. The reservoir 14 may hold a single dosage amount or multiple fluid dosage portions forming a single dosage amount for the final coating of one needle. The final microneedle coating dosage in the latter case is determined by x number of coating fluid portions repetitively filled into the reservoir 14 under control of control 12 and valve 20. In the multiple portion embodiment, the corresponding needle 6 then being coated is caused to be immersed into the reservoir 14 by the xyz positioning system via control 12 a predetermined number of times until all of the predetermined amount of reservoir 14 fluids are consumed to form the final coating thickness.

Valve 20 is opened and closed by control 12. Control 12 is computer operated in one embodiment in a dispensing system 10, which is commercially available and which embodiment will be described below. The control 12 in one embodiment may also automatically position reservoir 14 aligned with a selected needle 6 of the array 2 by the automatic xyz positioning system 13 included in the dispensing system 10. Control 12 also is programmed to control automatically the time that the valve 20 is open and thus meter the needed amount of liquid formulation 15′ supplied from the supply reservoir 16 to the needle coating reservoir 14 to complete one coating dosage on a single needle. An optional pump 22 may be used to supply the fluid from the supply reservoir 16 to the valve 20 via conduit 18.

It should be understood that the coated dosage on a needle represents a partial dosage of the at least one biologically active agent to be applied to a recipient. The combined coatings on all of the needles 6 of the array 5 form a full entire dosage of the at least one biologically active agent to be administered by the array 5. The liquid formulation 15′ may be supplied via optional pump 22 under operation of the control 12 in one embodiment or by gravity via fluid feed conduits 18, 18′ in a second embodiment. The reservoir 16 thus needs to be positioned appropriately relative to the position of the reservoir 14 for a gravity feed system.

In FIG. 1, the feed line 18′ feeds the reservoir 14 from the bottom providing a bottom fill inlet to the reservoir 14 for this purpose; however, this method of filling the reservoir 14 is optional as the reservoir may also be filled from the normally open reservoir top as shown in FIG. 2.

In FIG. 2, supply reservoir 16 is coupled to valve 20 by conduit 24. Computer operated control 12, via stored computer instructions including RAM and ROM, operates the valve 20 similar to the operation of control 12, FIG. 1. Identical reference numerals in the different figures correspond to identical parts. In this non-limiting embodiment, however, the output conduit 26 of the valve 20 feeds the liquid formulation to the microneedle receiving reservoir 28 via the top of the reservoir 28 rather than its bottom as in FIG. 1. Optional pump 22 or its equivalent, or gravity feed, also may be utilized.

In FIG. 3, representative reservoir 14 has an outside diameter D. The spacing between adjacent exemplary microneedles 6′, 6″ and 6′″ in all directions is L. The needles 6′, 6″ and 6′″ are identical and may be stainless steel or titanium having diameters W. The outside diameter D of the reservoir 14 is less than 2 L. This is so that the reservoir may fit in the interstitial space between alternate needles 6′ and 6′″ of the array 5 about the central needle 6″ being coated for all needles of the array 5, FIG. 1. The needles 6 have a diameter W that is smaller than the inside diameter of the reservoir 14 container 19 (based on a circular cylindrical reservoir 14) in order to be immersed into the liquid formulation 15 stored in the reservoir 14. The reservoir 14 receptacle 19 in one embodiment is circular cylindrical, but may be other shapes in other implementations as desired.

The xyz positioning system 13, in the alternative, may be a manually operated system. In this case, a microscope (not shown) is used to align visually the reservoir 14 with each microneedle 6 of the array 2, FIG. 1, via the xyz manual positioning system corresponding to system 13. The reservoir 14 is raised by the positioning system 13 to immerse the aligned needle 6 into the liquid formulation 15 sufficiently to use up all of the liquid formulation with a single or multiple immersions of a selected microneedle 6 as needed for a given implementation. Depending upon the amount of liquid formulation in the reservoir 14, a needle 6 may be inserted once or multiple times into that reservoir of the liquid formulation to provide a fully coated needle. Also, the reservoir 14 may, in certain implementations, be filled a number of times in order to provide a full dosage coating on the corresponding needle 6. Further, the reservoir bottom portion may contain a permanent predetermined amount of liquid formulation that will not be coated onto a needle 6. This is to permit the immersed needles to be spaced above the bottom wall 25 of the reservoir 14, FIG. 1 (and wall 27 reservoir 28, FIG. 2). This positioning of the needle relative to the reservoir bottom wall is controlled by the positioning system 13.

An XYZ positioning system 13 in an automatic mode is operated by the programmed control 12 which selectively and accurately positions the reservoir 14 in predetermined horizontal and vertical X, Y, and Z positions to manipulate the reservoir 14. This action immerses the selected microneedle 6 of the array 5 for coating. The dispensing system 10 may be a commercially available system manufactured by ED corporation such as its Ultra TT Automation Series, shown for example in FIGS. 9-11, and may also include its 741 series dispensing valves, shown for example in FIGS. 9 and 10, described below. The control 12 manipulates the reservoir 14 in any desired direction and distance to the needed accuracies in the X, Y and Z directions to align the corresponding reservoir 14 with each selected needle 6. The microneedles 6 are immersed into the liquid formulation 15 of the so positioned reservoir 14 to a desired depth in the fluid to consume the fluid fully in this embodiment, either with a single immersion or multiple immersions according to a given implementation.

The syringe needle 30, FIG. 9, forming the receptacle 19 of the reservoir 14, FIG. 1, may be of the type used, for example, in an embodiment of a commercially available dispensing system 54, FIG. 11. The liquid formulation reservoir 14 receptacle 19 of FIG. 1, more particularly, may be formed by a prior art hollow syringe needle 30 of fluid dispensing device 32, FIG. 9. The device 32 comprises an air cylinder 34, which may be stainless steel, a fluid receiving body 36, which also may be stainless steel, having a chamber 38 for receiving the liquid formulation from reservoir 16 (FIG. 1) to be dispensed to the needle 30. Device 32 also includes a fluid supply line 40 for supplying the liquid formulation to the fluid receiving chamber 38 of the syringe body 36.

Device 32 includes an inlet fitting 42 for supplying the liquid formulation from line 40 to the syringe chamber 38. The liquid formulation is dispensed from chamber 38 via needle 30 which forms the reservoir receptacle 19 of the reservoir 14, FIG. 1, for example. The needle 30 in this case is loaded with the liquid formulation, which is not forced out of the needle 30, but stored therein to form the reservoir such as reservoir 14, FIG. 1. The device 32 further comprises a pressurized air line 44 for providing pressurized air to a piston (not shown) in cylinder 34, which piston forces the liquid formulation from the chamber 38 into the needle 30 for storing the liquid formulation in the hollow syringe needle 30. The device 32 also includes an adapter 33 for attaching the needle 30 to the body 36 in fluid communication with the chamber 38. The adapter 33 is arranged to be secured releasably to the body 36 and is interchangeable with other adapters for receiving needles such as needle 30 of different dimensions. That is, different size needles 30 forming reservoirs of different capacities corresponding to microneedles of corresponding different dimensions may be used with the corresponding adapters 33.

The dispensing device 32 may operate millions of cycles without maintenance. The liquid formulation is applied to needle 30 with accurate, close repetitive control via a computer programmed control in the system such as system 54, for example, which may provide the control 12, FIG. 1. The needle 30 stroke distance in direction 35 is set by a stroke setting device 37, FIG. 9, which is rotated in directions 39. The stroke distance controls the depth of penetration of the corresponding microneedle into the liquid formulation of the reservoir, the microneedle being fixed in position at the time of its immersion into the reservoir which is displaced relative to the microneedle.

The device 32, FIG. 9, represents the valve 20, FIG. 1, which is operated by control 12 as commercially available as control 41, FIG. 11, for operating the device 32 of FIG. 9. In FIG. 1, the pump 22 schematically represents the piston (not shown) in the device 32, FIG. 9, which selectively periodically forces the liquid formulation into the needle 30 in periods and amounts as determined by the control of system 54, for example, or other similar commercially available system that may be used.

In FIG. 10, a representative control panel 46 of a commercially available dispensing system for operating control 12 (FIG. 1) includes function indicators 46 which include power, run, setup and cycle modes of the control 12 whose detailed operation is not described herein because this is a commercially available system. A pressure/time toggle 48 and an emergency stop switch 50 are also provided. The display 52 displays various parameters for operating the dispensing device 32, FIG. 9, including set time, timer bypass, pressure of air in air line 44 (FIG. 9), a teaching program stored in computer memory (not shown), a test cycle operated by the control 12, a purge mode for purging the liquid formulation from the system and a reset control for resetting the device 32. There is a push button adjustment of a valve open time which controls the amount of liquid formulation supplied to the needle 30, FIG. 9. The deposit size determined by controlling the amount of liquid formulation supplied to the needle 32 (FIG. 9) and thus the reservoir 14 (FIG. 1) is programmed by pressing a PROGRAM button (not shown) in the setup mode. This commences selection of the amount of liquid formulation supplied to the reservoir 14, FIG. 1 (needle 30, FIG. 9).

FIG. 11 depicts an exemplary automated xyz dispensing system 54 with integrated controllers for operating two dispensing devices 32 as shown as compared to manually operated systems or a single device 32 in other embodiments of other commercially available systems. The system 54 has an electronically controlled xyz positioning platform 56 for aligning optionally a microneedle array in an alternative embodiment to the reservoir needles of the two devices 32. The various gauges, display and control knobs and buttons on the front face of the control unit 41 are explained in corresponding literature available with the commercially available system. The amount of fluid deposited into a reservoir needle 30 (FIG. 9) and thus reservoir 14 (FIG. 1) and the placement of the fluid deposit into the reservoir 14 into alignment with a selected microneedle 6 (FIG. 1) are programmed into the system of FIG. 11 with an input device such as a personal data assistant (PDA) 56 or teaching pendant.

A liquid formulation 15 is fed from the supply reservoir 16, FIG. 1, to the receiving reservoir in an amount sufficient for the production of at least one layer of coating on the microneedle 6, FIG. 1, but not to exceed the desired dose of biologically active material for the coating on a microneedle. The microneedle is then brought into a temporary contact with the coating liquid formulation either by displacing the reservoir 14 or the microneedles or both, to produce a layer of coating on each microneedle 6. In one embodiment, the process is repeated until the coating fluid in the reservoir is consumed and a multilayer coating containing the desired dose of biologically active material is created on each microneedle 6.

Thus, after the liquid formulation 15 in the reservoir 14 is consumed, the amount of the biologically active compound deposited on each microneedle 6 of the array of needles is predetermined by this consumed amount to form the correct desired dosage for that needle 6. The coating amount thus is not controlled by the number of contacts or dips, but only by dispensing a precise volume of the liquid formulation to each microneedle. This approach prevents overdosing of the biologically active compound, and thus undesirable side effects, and also minimizes the development and validation work needed to establish a manufacturing process. The disclosed method of coating the microneedles can be performed one or more times for a given microneedle, when higher doses of biologically active compound are desirable, and multiple reservoirs of the liquid formulation of the coating fluid may be required.

The volume of the liquid formulation fed to the microneedle is controlled at all times and thus the dose of biologically active compound for each microneedle is controlled accurately as well. Also, a liquid drug or other biologically active compound containing formulation is not exposed to ambient atmospheric air for an undesirable length of time. This insures minimizing undesirable changes in the drug content, and in the viscosity of the coating fluid formulation, due to the drying or evaporation of the liquid formulation in the reservoir 14 formulation or the equivalent of reservoir 14 in other embodiments.

According to the method of the herein disclosed embodiments, the dose of the biologically active compound deposited on the microneedles is calculated as follows:

D_{b}=f×C_{b}×ΔV, (1)

wherein D_{b} is the dose of biologically active compounds on one microneedle, f is the number of feeds of the liquid formulation to the applicable fluid reservoir, C_{b} is the concentration of a biologically active compound, and ΔV is the volume of a single feed.

The reservoir containing the liquid formulation, such as reservoir 14 shown in FIG. 1, can be of any geometrical form and comprise an opening 9, FIG. 1, that allows for the contact between each microneedle 6 and the liquid formulation 15 containing the biologically active material. In the preferred embodiment, the coating reservoir 14 has a cylindrical shape. In the most preferred embodiment, the coating reservoir 14 is of the shape similar to or conforming to the shape of the microneedle. The cylinder interior dimensions of the reservoir receptacle 19, FIG. 3, allow the microneedle to be immersed into contact with the liquid fluid formulation. In one embodiment, the internal radius of the cylinder may be smaller than approximately the width w of the microneedle (FIG. 3) and the outside radius of the reservoir cylinder does not exceed the shortest distance between the microneedles, and most preferably, the outside radius is about half of the shortest distance between the microneedles along their length dimension L, FIGS. 7 and 8.

In one embodiment shown in FIG. 7, the length L₁ of the cylinder 19 of the reservoir 14 exceeds at least one third of the microneedle 6 length L, and in another embodiment, two thirds of the microneedle length. The volume of the liquid formulation 15 in the reservoir 14 in one embodiment exceeds the volume of the single feed (ΔV). In yet another embodiment, the reservoir 14 includes a physical cover 66, FIG. 7a, containing an orifice 68 to allow the insertion of the microneedles 6 into the reservoir interior into the liquid formulation 15, but preventing the substrate 4, FIG. 7, of the microneedle from contacting with the coating liquid formulation 15. The coating reservoir can be made of a variety of materials compatible with the liquid formulation of the biologically active compound, such as stainless steel, titanium, glass, or plastic.

It should be understood that a coating reservoir (not shown), in a further embodiment, may accommodate multiple microneedles, such as an entire array, for example. In this case, the amount of the liquid formulation fluid fed to the reservoir 14 (f in the equation 1) is multiplied by the number of microneedles in the array. Subsequently, to obtain the dose of biologically active compound coated on the single microneedle (D_{b} in equation 1) according to equation 1, the product f×C_{b}×ΔV, is divided by the number of microneedles in the array. The coating reservoir in this case has a physical cover such as cover 66, FIG. 7a, comprising an array of orifices corresponding to the number and position of the microneedles in the array. Such a cover allows the contact of the liquid formulation in the coating reservoir with the microneedles, but does not allow the substrate supporting member of the needle array to contact the formulation. This avoids or minimizes the loss of biologically active fluid. The needles of the array thus together form the desired total dosage to be administered by the needle array. Thus the dose on each needle in practice forms a partial dose which when combined with all needles of the needle array forms the final desired dosage to be administered.

The contact time between the microneedle and liquid formulation may vary depending on the liquid formulation to be applied to the microneedle, the fluid viscosity, the geometry of the microneedle, stability of the biologically active component, and the solubility of the previous layer of the coating. In one embodiment, the contact time of the liquid formulation with the microneedle is between 1 and 10 seconds. The number of repetitive contacts between the microneedle and the liquid formulation required for the full deposition of the coating onto the microneedle is dependent on the characteristics of the coating reservoir, the dose of drug or biologically active compound to be deposited, and properties of the formulation. In one embodiment, the number of such repetitive contacts is equal to the number of contacts needed for the full consumption of a single feed of the liquid formulation to the reservoir, such as reservoir 14, FIG. 1. Alternatively, the number of contacts may exceed the number of contacts needed for the full consumption of a single feed. Generally, the extent or the depth of contact remains the same during the coating process. Alternatively, the depth of contact can be varied, so that the thickness of the coating across the microneedle is varied.

In one embodiment, the contact between the microneedle and liquid formulation 15 is followed by drying of the coating fluid coating on the microneedle(s). The drying process may be conducted by exposing the microneedle coating(s) to the air at ambient temperature. Alternatively, drying may be performed in a controlled environment, such as at elevated temperature, or in a controlled humidity, or in a nitrogen atmosphere. In one embodiment, the drying time is between 1 and 60 seconds. In another embodiment, the drying time is between 1 and 10 seconds. Of course, this drying time is contingent upon the liquid formulation and the environment in which the drying is occurring.

In order to supply the required feed of liquid formulation to the coating reservoir, various types of dispensing and microdispensing systems, such as mechanical, air, gravity, or vacuum driven systems can be used. Such systems may generally contain a valve, or similar device, to control the volume of the liquid formulation containing biologically active material being fed to the coating reservoir. In one embodiment, the feeding of the liquid formulation including at least one biologically active agent may be periodic with a rate that can exceed the consumption of the coating liquid formulation in the microneedle coating step.

In yet another embodiment the feeding of liquid formulation may be continuous with a feed rate that does not exceed the consumption of the liquid formulation. In another embodiment, the coating reservoir may be in continuous fluid communication with the supply reservoir, for example, in a gravity feed system wherein the source reservoir is positioned to feed the desired amount of liquid formulation automatically to the reservoir. In this case, as the source reservoir fluid is depleted, a control system (not shown), such as a computer operated control, is provided to monitor continuously the fluid level in the source reservoir to insure it is at the desired position necessary to insure the coating reservoir receives the proper predetermined level of liquid formulation therein. Also, the amount of liquid formulation in the coating reservoir may be monitored by sensors (not shown) via a control to be sure the fluid is at the predetermined level corresponding to a given dosage prior to immersion of a microneedle.

In a further embodiment, the coating liquid formulation is fed to the coating reservoir through an opening in the coating reservoir, which feeding may be controlled by a computer or a manually controllable valve to provide the desired feed volume of the coating fluid to the reservoir. In yet another embodiment, the coating reservoir has no separate supply opening. The coating liquid formulation is supplied via a conduit from the supply reservoir to the coating fluid reservoir through the top of the coating liquid reservoir which is normally open to the ambient atmosphere using the microdispensing system described in FIGS. 1, 2, and 9-11 above. When the feed of the liquid formulation to the coating liquid reservoir is completed, the liquid feed to that reservoir is halted until the liquid in that reservoir is consumed as described above.

To provide flow of the coating liquid formulation to the selected microneedle(s) from the coating liquid formulation source to the coating liquid reservoir, a variety of positioning and micropositioning systems such as the types described above herein, or other commercially available systems, may be utilized. For example, in one embodiment, a manual three-dimensional (XYZ) micropositioning system and stage can be used for positioning the microneedles and/or the coating liquid reservoir(s) according to a given implementation. In a most preferred embodiment, automated or motion control, such as computer software controlled, positioning is employed as described herein.

In FIG. 4, in a further embodiment, system 70 comprises an array 72 of microneedles 74 to be coated with a coating liquid formulation and attached to a substrate 76. The needles 74 are substantially identical and are in a symmetrical array wherein the spacing between the needles is substantially identical throughout the assembly. The needle array 72 is fixed in position.

A like array 78 of coating liquid reservoirs 80 are secured to a support 82. The reservoirs 80 may comprise reservoirs similar to the needles 30, FIG. 9, or other similar reservoir receptacles for receiving and coating the microneedles 74. The array 78 is substantially the same in dimensions between reservoirs in two orthogonal dimensions. Thus the needles 74 may all be inserted simultaneously into and immersed in a coating liquid formulation stored in each reservoir 80. Each reservoir 80 receives an identical amount of coating liquid formulation from the supply reservoir 84 via conduit system 86. The needles 74 are immersed into their corresponding reservoirs simultaneously.

Conduit system 86 comprises a control 88 which opens and closes valve 90 in conduit 92 to meter the correct predetermined amount of coating liquid formulation to a corresponding reservoir 80. Control 88 also includes a programmed computer controlled xyz positioning arrangement. Conduit 92 is coupled selectively to each reservoir 80 via a corresponding reservoir input conduit 94 in an array 96 of conduits. Conduit 92 also comprises conduit section 98 which is displaceable in orthogonal two dimensional xz directions. Section 98 is displaced to couple selectively the conduit 92 to a selected one of conduits 94. For example, the section 98 may comprise a displaceable dispensing device such as needle device 32, FIG. 9. The section 98 includes in this case a dispensing needle such as needle 30 or the like which is coupled sealingly to a selected conduit 94 by a sealing pliable valve flap and the like. The reservoirs 80 in array 78 in turn may comprise an array of needle-like receptacles similar to receptacle 19 formed by needle 30.

The conduits 94 are prefilled with coating liquid formulation prior to filling the reservoirs 80. The reservoirs 80 also are filled partially at all times with the same amount of coating fluid. Pressurized fluid from the dispensing conduit system 86 under control of control 88 fills each reservoir 80 with an identical amount of coating fluid. The length of the conduits 94 may be relatively short, the drawing being not to scale for purposes of illustration. The conduits may be at any desired convenient orientation, the orientation of the figure being given only for illustration. For example, the conduits 94 need not be at right angles as shown, but may comprise short linear vertically oriented sections engaged in fluid communication by section 98 of the conduit system 86. In the alternative, the conduits 94 may be omitted and the conduit system 86 may engage the reservoirs in direct fluid communication to fill directly each reservoir 80 from section 98. The section 98 is displaced in an appropriately oriented xz direction so to engage the reservoirs 80.

The control 88 injects the same amount of fluid into each of the reservoirs 80. It does this by opening the valve 90 for a predetermined time period and applies the same pressure to the fluid in the conduit section 98 to inject the fluid into the reservoirs 80. All conduits, for example, may be vertical and aligned vertically with the reservoirs 80.

In system 70, all microneedles are coated simultaneously providing for a more rapid coating arrangement than a system that coats the microneedles one at a time.

In the alternative to a single section 98 and conduit 92 that is displaced to position section 98 in alignment with each conduit 94 as discussed above, the sections 98, valves 90 and conduits 92 may be arranged in a further embodiment in an identical array (not shown) corresponding to the array of conduits 94 and array of reservoirs 80 and coupled to the array 78 of reservoirs 80 simultaneously. In this embodiment, there is a corresponding array of valves 90, each valve 90 being associated with a corresponding conduit section 98 of the array of conduit sections. Control 88 opens and closes these valves 90 in the array sequentially to apply the same amount of coating fluid formulation to each reservoir 80.

The liquid formulation in the conduits 92 in this case is pressurized to cause an identical amount of liquid formulation to be injected into each conduit 94 when the valve 90 is opened and thus into the corresponding reservoir 80. Control 88 controls the operation of the array of the valves 90 in the specified sequence. Such operation of the valves 90 in sequence increases the speed in which the reservoirs 80 can be filled. The timing of the valve opening and pressure can be determined empirically and controlled by a programmed controller (not shown). Sensors (not shown) also can be used to sense the amount of fluid in each reservoir such as optical sensors used in conjunction with optically transparent reservoirs 80 or flow sensors that can be used to sense the liquid formulation flowing in the conduits such as conduit 92 or 94, for example.

FIG. 6 illustrates another embodiment wherein the coating liquid formulation is filled in the coating reservoirs from the top. This is somewhat similar to the embodiment of FIG. 2. Needle coating system 100 comprises a microneedle array assembly 102 comprising an array 104 of microneedles 106 secured to a substrate 108. The assembly 102 is releasably attached to a movable platform 110 of an xyz positioning system 112 that is part of the system 100. The system 112 is operated by programmed control 114. The needles 106 of the array 104 are identical and are in a symmetrical identical spacing as are the microneedles in all of the embodiments disclosed herein.

An array 116 of reservoirs 118 is attached to a further xyz positioning system 120 via support 122. The reservoirs 118 may be identical to reservoirs 14 described above in connection with FIG. 1 except they are filled from the top, and not the bottom. The control 114 operates a pump 124 via line 130. Pump 124 receives the coating liquid formulation from the supply reservoir 126 via conduit 128. The control 114 also operates valve 132 to meter the coating liquid formulation via conduit 134 to selected ones of the reservoirs 118 of the array 116. It should be understood that the pump 124, valve 132 and the conduit 134 in one embodiment may be represented by the device 32, FIG. 9 and the control 114 may be represented by the control of system 54, FIG. 11. The xyz positioning system 112 may be represented by the platform 56 controller of the system 54, FIG. 11. The xyz positioning system 120 for positioning the reservoirs to receive the coating liquid formulation from the conduit 134 may also be controlled by an appropriately programmed system such as the controller of system 54 or other xyz positioning controllers that are commercially available.

In operation, the reservoirs 118 of the array 116 are filled with the predetermined amount of coating liquid formulation.

In one non-limiting embodiment, the asperities, microprojections, or microneedles are solid. In another non-limiting embodiment, the asperities, microprojections, or microneedles are hollow.

While the microneedle embodiment (described below) may be employed, other systems and apparatus that employ tiny skin piercing elements to enhance intradermal agent delivery also are contemplated, as disclosed in U.S. Pat. Nos. 5,879,326, 3,814,097, 5,250,023, 3,964,482, Reissue U.S. Pat. No. 25,637, and PCT Publication Nos. WO 96/37155, WO 96/37256, WO 96/17684, WO 97/03718, WO 98/11937, WO 98/00193, WO 97/48440, WO 97/48441, WO 97/48442, WO 98/00193, WO 99/64580, WO 98/28037, WO 98,29298, and WO 98/29365; all incorporated herein by reference in their entireties.

It is not intended that the present invention be limited to a precise geometry or topology of the microneedles. In one embodiment, the microneedles are defined by a plurality of surfaces sloping upwards from a relatively broad base to a tip (e.g. a pyramidal shape). In another embodiment, the microneedles have a generally conical-shaped body (e.g. a single curved surface).

Furthermore, within the scope of the present invention, the asperities, such as microprojections and microneedles, in one embodiment, may include one or more indentations and/or barbs, which aid in retaining the formulation on the asperities.

It is not intended that the present invention be limited by the precise dimensions of the microneedles. In one non-limiting embodiment, the microneedles described herein have a microneedle height to width (measured at the base) ratio of between 1.2 and 2.0 (and in one embodiment between 1.4 and 1.7), and the structure is predominantly solid, rather than hollow. These factors contribute to the force required to penetrate human skin being smaller than that required to break the penetrating elements. This applies to both single microneedles and various microneedle arrays, for which penetration forces are different depending on the number of penetrating elements, their height, and the spacing between adjacent microneedles.

In one non-limiting embodiment, a prototypical microneedle has a diameter of between 200 and 500 microns (and in another embodiment, between 300 and 400 microns) at its broad end (e.g., at the base), and tapers to a sharp tip or chisel edges with a somewhat smaller diameter at its other end. The diameter of the tip may, for example, be in the range from about 50 microns to about 1 micron.

In one non-limiting embodiment, the microprojections or microneedles have a length (or height) up to 1000 microns, and in one embodiment between 100 microns and 1,000 microns, and in another embodiment less than 700 microns, but more than 250 microns. In yet another embodiment, the microneedles have a length or height of from 300 microns to 600 microns In another embodiment, the microneedles have a height of between 550 and 650 microns (such as, for example, between 580 and 620 microns) with a height to width ratio of between 1.5 and 1.7. The microprojections may be formed in different shapes, such as needles, blades, pins, punches, and combinations thereof. In one embodiment, the microneedles are pyramidal in shape (e.g., having between 6 and 12 sides, and in one embodiment, eight sides).

The asperities, microprojections, or microneedles may be constructed from a variety of materials, including but not limited to, metals and metal alloys, such as titanium, stainless steel, nitinol, gold, silicon, silicon dioxide, ceramics, and polymers, including but not limited to synthetic and natural, water soluble and water insoluble, biodegradable, organic, and organometallic.

Although not limited solely hereto, suitable arrays of asperities can made by the growth of elongated cylindrical crystals by a vapor-liquid-solid process; growth of polycyanoacrylate fibers from small deposits of catalyst material; MEMs technology of the sort utilized in the semiconductor electronics industry; removing, by dissolution, fracture, or decomposition, the matrix from a composite that contains acicular particles; and in many other ways known in the art.

In one non-limiting embodiment, the asperities are microneedles which are anisotropically etched MEMs microneedles in silicon.

In one non-limiting embodiment, the solid microneedles are fabricated in a crystal silicon material suitable for use in the administration of the various preparations discussed herein.

In another non-limiting embodiment, the asperities, microprojections, or microneedles are made from metal, and in one non-limiting embodiment, the metal is titanium.

The metal asperities, microprojections, or microneedles can be prepared by a variety of techniques including but not limited to laser cutting, or chemical etching, including inductively coupled plasma dry etching. The asperities, microprojections, or microneedles then can be electropolished to provide a smooth surface or may be anodized, or otherwise surface modified to create the desired surface chemistry. In a non-limiting embodiment, such asperities, microprojections, or microneedles have a length of from about 100 microns to about 1,000 microns. In another non-limiting embodiment, the microneedles have a length of from about 300 microns to about 600 microns. In a non-limiting embodiment, the microneedles are produced in the form of arrays. One such arrangement of microneedles is shown in FIG. 5. In FIG. 5, device 60 comprises a substrate 62. An array 64 of microneedles is attached to the substrate 62. The array 64 shown in FIG. 5 includes 63 microneedles.

An array of microneedles may contain any number of microneedles. In a non-limiting embodiment, the array contains at least 50 microneedles. In such arrays, the microneedles may be attached to the base or substrate at an angle to the base or substrate. In one non-limiting embodiment, the microneedles are attached to the base or substrate at a right angle (90°) to the base or substrate. The base or substrate, in a non-limiting embodiment, may be made of the same material as the microneedles, such as titanium, or, in another non-limiting embodiment, be made of another material, such as plastic, rubber, or metal.

The coated microneedle devices are useful in transporting biologically active agents across the biological barriers in humans, animals, or plants. These barriers generally include skin or parts thereof, such as epidermis, mucosal surfaces, blood vessels, and cell membranes, In one embodiment, the microneedle devices are useful for the delivery of biologically active compounds into human skin, such as the epidermis. They typically contain skin piercing elements to penetrate stratum corneum and can be applied with the applicator to maintain the desired pressure and time of the application. In another non-limiting embodiment, the microneedles deliver the at least one biologically active agent to the dermis.

Without limiting the invention in any manner to any particular mechanism, it is believed that the biologically active agents or drug(s) is (are) delivered by microporation of the stratum corneum, and polyphosphazene polymer-drug deposition within the patient's skin and subsequent dissolution or erosion of the polymer. The drug becomes thereby bioavailable; it can dissolve and diffuse to the biological target, or alternatively, it can remain at the site of administration. Micropores are made into the stratum corneum by means of a microneedle array penetration, which optionally can be enhanced further by applying energy in the form of ultrasonic, heat and/or electric signals across or through the skin.

In a non-limiting embodiment, coated microneedle devices of the present invention are applied to the skin for a period of time required for the coating to dissolve, disintegrate, erode, degrade, swell, or undergo other physical chemical, or biological changes to release the at least one biologically active agent. In a non-limiting embodiment, the coating is water soluble so that it may dissolve quickly upon contact with body fluids. In one non-limiting embodiment, the dissolution time is between 1 second and 60 minutes. In another non-limiting embodiment the dissolution time is between 1 and 600 seconds.

It is not intended that the present invention be limited by the nature of the substrate comprising the microneedles. In one non-limiting embodiment, the microneedles are formulated from a polymer. In another non-limiting embodiment, the microneedles are made with a mold. In yet another non-limiting embodiment, the microneedles are etched out of a silicon substrate. In a further non-limiting embodiment, the silicon microneedles are solid, and the formulation of the present invention is deposited on the microneedles.

It also is not intended that the present invention be limited to inflexible microneedle arrays. Indeed, embodiments of flexible microneedle arrays are contemplated. In one embodiment of a flexible microneedle, the present invention contemplates separating microneedles into individual “islands” by cutting into (and even through) the substrate so as to define such islands or regions separated by channels or streets (which can be, in one embodiment, filled or partially filled with polymer or drug). In one embodiment, the present invention contemplates mounting the substrate onto an adhesive material (e.g. adhesive tape) and dicing or cutting through the substrate to generate flexible arrays. In this manner, the risk of breakage when pushing against the back of the silicon substrates, when applying the patch to the skin, is reduced.

Various features can be added to the microneedle arrays to assure proper delivery. In one embodiment, the present invention contemplates the use of a plastic or otherwise elastomeric device positioned above the array relative to the skin (or attached or incorporated into the substrate or upper layer) that snaps into place once pressure is applied against the patch to push and keep the array of microneedles in the skin while the patch is on (to make sure needles are inside the skin and to avoid the need for an applicator in the final product, which is fully disposable in this embodiment). In one embodiment, the elastomeric element takes a first form prior to administration and then takes a second form after application of pressure. In other words, the elastomeric element (which can be arched, curved or generally U-shaped) undergoes a shape change or deformation upon receiving the pressure from pushing the array into contact with the skin (e.g. from concave to convex).

The present invention, as mentioned above, also contemplates methods of administering at least one biologically active agent. In one embodiment, the present invention contemplates a method of administering at least one biologically active agent, comprising: providing a subject and the delivery device described above; and contacting said subject with said delivery device under conditions such at least a portion of said biologically active agent is released from said device. The term “subject” includes human and non-human animals. In the case of humans, the term includes more than patients. The term also includes healthy, asymptomatic recipients. In one embodiment, said contacting comprises piercing the subject's skin with said asperities such as microprojections or microneedles.

The present invention also contemplates, in one embodiment, a device for delivering at least one biologically active agent comprising: a substrate having a back surface and a front surface; and a plurality of solid microneedles extending upwards from the front surface of the substrate, the microneedles coated with the formulation of the present invention, said formulation comprising at least one polyphosphazene polyelectrolyte and at least one biologically active agent.

When in an array, the density of the microprojections is, in one non limiting embodiment, at least 10 microprojections/cm², in another embodiment, at least 200 microprojections/cm², and, in some embodiments, at least 1000 microprojections/cm². In one embodiment, each microneedle is spaced (when measured center to center with another microneedle) between 300 microns and 2.7 mm apart. In one embodiment, the spacing is approximately three times the height of the microneedle, i.e. for a microneedle that is 600 microns (plus or minus 200 microns) in height, the spacing may be 1.8 mm, while for a microneedle that is 900 microns in height, the spacing may be 2.7 mm, while for a microneedle that is 300 microns in height, the spacing may be 900 microns. It is to be understood that the present invention is not to be limiting to any particular density of microneedles.

The invention now will be described with respect to the following example; it is to be understood, however, that the scope of the present invention is not intended to be limited thereby.

### EXAMPLE 1

### Preparation of Microneedle Coatings Containing Bovine Serum Albumin (BSA)

In this example a polyphosphazene polyelectrolyte, poly[di(carboxylatophenoxy)phosphazene], sodium salt (PCPP) was used to form a coating containing BSA on titanium microneedles. In separate experiments a viscosity enhancer—water-soluble non-polyphosphazene polyelectrolyte, carboxymethylcellulose sodium salt (CMC) was also used for comparative purposes. The concentrations of CMC solutions were such, that their solution viscosities in 0.1×PBS were the same or higher than the viscosity of PCPP solution in 0.1×PBS (Table 3).

TABLE 3
Characteristics of coating forming polymers.

| | | Solution | |
|---|---|---|---|
| | | Concentration, % | Solution |
| | | (wt./vol.) | Viscosity*, cps |
| Experiment No. | Polymer | (in 0.1xPBS) | (in 0.1xPBS, 24° C.) |
| 1 | PCPP | 0.5 | 5.1 |
| 2 | CMC | 0.8 | 5.1 |
| 3 | CMC | 1.5 | 13.8 |

*measured using calibrated Ubbelohde viscometer UBB-1C, VWR,

Three coating formulations were prepared, all containing 5% (wt./vol.) of bovine serum albumin in 0.1×PBS solution, but with different polymers and its concentrations: 0.5% (wt./vol.) of PCPP was used in Experiment 1, and 0.8 and 1.5% (wt./vol.) of CMC were used in Experiments 2 and 3, respectively.

The coating process was performed using 2400 Series Digital Time-Pressure Dispenser (EFD, Inc., East Providence, R.I.), containing a 1 mL barrel reservoir equipped with a PTFE lined dispensing tip (5125TLCS-B, EFD, Inc., East Providence, R.I.). A stereo zoom microscope (STZ-45-BS-FR), with a 2.0 megapixel 1616×1216 digital camera (Caltex Scientific, Irvine, Calif.) and an AM-311 Dino-Lte digital microscope with adjustable magnification from 10× to 200× (BIGC, Torrance, Calif.) were used to monitor the coating process.

An array containing 50 titanium microneedles (length-600 μm) was used in the coating process. A microneedle array was attached to lower surface of a horizontal stage on an X-Y-Z-micropositioning system using double-sided adhesive tape and the dispenser was set up in a vertical position on a ring stand. Using the X-, Y-, Z-control knobs, the microneedles were aligned over the dispenser tip to assure proper insertion before the coating. The dispenser was purged with the formulation to remove air bubbles and to fill the tip up to level the liquid with the dispenser tip. Then a feed of a formulation was supplied corresponding to a single pulse and resulting in the formation of a meniscus over the dispenser tip. The microneedle of the array then was brought into contact with the liquid, removed from the liquid, and air dried. The process then was repeated and the total number of contacts (dips) was counted during the experiment. A series of microneedle arrays were coated with each formulation and within each series, arrays with varied number of dips were obtained.

The coating then was analyzed for the protein loading. The microneedle array was rinsed with 0.2 ml of 0.1× phosphate-buffered saline (PBS) to dissolve the coating and the protein loading was quantified using size exclusion chromatography—Hitachi LaChrom Elite HPLC system (Hitachi High Technologies America, Inc., San Jose, Calif.), equipped with L-213OHTA pump with degasser, L-2200 autosampler, L-2455 Diode array detector, L-2490 refractive index detector, EZChrom Elite Stand-Alone Software for Hitachi LaChrom Elite HPLC, and Ultrahydrogel 250 column with a guard column (Waters, Milford, Mass.). 0.1×PBS, containing 10% acetronitrile was used as a mobile phase with a flow rate of 0.75 mL/min and an injection volume of 0.095 mL. Calibration curves for determining the amount of protein in the analyzed samples were obtained via serial dilutions of the coating formulation. The results were plotted for each series as the amount of protein detected on the microneedle by HPLC versus the number of dips applied to the array (FIG. 12).

The results demonstrate that polyphosphazene polyelectrolyte, PCPP, is capable of forming a protein-containing microneedle coating. The rate of coating formation is significantly higher than the other tested non-polyphosphazene polyelectrolyte, CMC (FIG. 12). This phenomenon cannot be explained by the viscosity enhancing properties of the polyphosphazene itself, because the viscosity enhancing properties of CMC at tested concentrations in 0.1×PBS were the same or superior (Table 3).

### Examples of Polyphosphazene Polyelectrolytes as Immunostimulating Compounds for Intradermal Immunization

### EXAMPLES

### Example 2

### Preparation of Microneedles Containing HBsAg and PCPP

Microneedles containing solid state formulation of antigen, Hepatitis B surface antigen (HBsAg) were prepared for in vivo immunization experiments. This was achieved through deposition of antigen containing formulation on the surface of metal microneedles using a micro dip-coating process. Polyphosphazene polyelectrolyte, PCPP, was employed as a coating forming polymer to bind the antigen to microneedles.

Titanium microneedle arrays, each containing 50 microneedles of 600 μm in length, were prepared by chemical etching. They were washed in an ultrasonic bath cleaner with the following solvents: deionized water, isopropanol, deionized water, ethanol, isopropanol.

The coating formulation contained 1% (w/v) of PCPP, disodium Salt (Sigma, St Louis, Mo., USA), purified by multiple precipitations in aqueous sodium chloride, 0.3% (w/v), HBsAg adr Rec (Fitzgerald Industries International, Inc., Concord, Mass., USA), and 0.1% (v/v) polyoxyethylene (20) sorbitan monolaurate (Tween-20) (TCI America, Portland, Oreg., USA) in 0.6× Dulbecco's Phosphate Buffered Saline (DPBS) (Sterile, without Calcium or Magnesium, Lonza, Walkersville, Md.). The stock solutions of PCPP and Tween-20 were filtered through sterile 0.45 μm Millex syringe filters before mixing with HBsAg.

The coating system was equipped with a 50 micro-well coating reservoir (80 □I volume), an X-Y-Z micro-positioning system, a drying reservoir, and an optical microscope. The formulation was fed to the reservoir using a syringe with a modified steal plunger and using a Genie Plus syringe pump (Kent Scientific, Torrington, Conn., USA).

A microneedle array was secured on the array holder and then attached to the X-Y-Z micro-positioning system using alignment pins and holders. Using the micro-positioning system, the coating procedure was performed by submerging the microneedle arrays into the coating reservoir and immediate removal from the reservoir, followed by a drying step in which the arrays were purged with anhydrous nitrogen gas. Multiple coating cycles were performed to achieve the desired dose of the antigen, and the level of the formulation in the coating reservoir wells was restored to a pre-set level before every coating cycle.

A stereo zoom microscope (STZ-45-BS-FR), with a 2.0 megapixel 1616×1216 digital camera (Caltex Scientific, Irvine, Calif.) was used to monitor the coating process.

The analytical characterization of coated arrays was performed on representative arrays. 20% of prepared arrays were subjected to analysis to determine the dose of HBsAg and the amount of PCPP on the array. The analysis was conducted using UV/V is Spectrophotometry and size-exclusion HPLC. Serial dilutions of coating formulation with known amounts of antigen and polymer were used to create calibration curves for both methods.

Coated arrays, which were randomly selected for the analysis, were processed by placing each one of them in a separate plastic weigh boat and dissolving the coating in 1 mL of 0.1×PBS.

Spectrophotometric analysis was performed using the HITACHI U-2810 Spectrophotometer and the optical density was measured at 280 nm.

HPLC analysis was conducted using the Hitachi LaChrom Elite HPLC system (Hitachi High Technologies America, Inc. San Jose, Calif.), equipped with L-213OHTA pump and degasser, L-2200 autosampler, L-2455 Diode array detector, and L-2490 refractive index detector. An Ultrahydrogel 250 size exclusion column (Waters, Milford, Mass.) was used for separation. 0.1×PBS, containing 10% acetonitrile was employed as a mobile phase and the flow rate was set to 0.75 mL/min. The injection volume was 0.095 mL. The results were processed using EZChrom Elite Software (Hitachi High Technologies America, Inc. San Jose, Calif.).

The results of the analysis are presented in Table 4.

TABLE 4
Analytical results for microneedle
arrays from Examples 2, 3, and 5.

| | |
|---|---|
| HBsAg, | PCPP, |
| μg/array | μg/array |

| | UV | HPLC | UV | HPLC |
|---|---|---|---|---|
| Example 1 | 11.1 ± 0.8 | 11.0 ± 0.7 | 35.7 ± 2.3 | 35.7 ± 2.1 |
| Example 2 | 4.8 ± 0.1 | 4.9 ± 0.1 | 30.9 ± 0.9 | 31.3 ± 0.8 |
| Example 4 | 10.1 ± 0.8 | 11.5 ± 1.3 | — | — |

### Example 3

### Preparation of Microneedles Containing Reduced Dose HBsAg and PCPP

The arrays were prepared as described in Example 2 except that HBsAg was used at a concentration of 0.15% (w/v) in the coating formulation. The results of the analysis are presented in Table 4.

### Example 4

### Comparative

### Preparation of Aqueous Formulation Containing HBsAg and PCPP

For comparative purposes, a solution containing HBsAg and PCPP was prepared for intramuscular injection as follows. 0.66 mL of 1 mg/mL of PCPP solution was added to 9.305 mL of sterile 1×DPBS solution. 0.035 mL of 5.66 mg/mL HBsAg solution was added and mixed well. 1 mL of the final solution contained 0.02 mg of HBsAg and 0.066 mg of PCPP.

### Example 5

### Comparative

### Preparation of Microneedles Containing HBsAg

For comparative purposes, the antigen, HBsAg, was coated as described in Example 2 with an inert film forming polymer—sodium carboxymethyl cellulose, CMC, instead of PCPP, to bind the antigen to the microneedles. CMC is a water-soluble anionic polymer and is included in the Inactive Ingredient Database of U.S. Food and Drug Administration for use in approved drug products for intradermal, intramuscular, and subcutaneous injections.

The coating formulation was similar to the one described in Example 2, except that it contained 1% (w/v) of USP/NF grade CMC (Aqualon® Sodium Carboxymethylcellulose, USP/NF grade, low viscosity, Hercules, Wilmington, Del., USA) instead of PCPP. The stock solution of CMC was filtered through sterile 0.45 μm Millex syringe filters before mixing with HBsAg.

The results of the analysis are presented in Table 4.

### Example 6

### Comparative

### Preparation of Aqueous Formulations Containing HBsAg for Intramuscular Immunization

For comparative purposes, a solution of HBsAg in 1×DPBS was prepared at a concentration of 20 μg/mL. A 1 mL aliquot (i.e. 20 μg dose) of this solution was used for intramuscular immunization per subject.

### Example 7

### Comparative

### Preparation of Aqueous Formulations Containing HBsAg for Intradermal Immunization

For comparative purposes, a solution of HBsAg in 1×DPBS was prepared at a concentration of 100 μg/mL. This solution was used for intradermal immunization by injecting four 50 μL aliquots to total 200 μL (i.e. 20 μg dose) per subject.

### Example 8

### In Vivo Immunization Experiments

In vivo immunization experiments were conducted in Land Race Cross pigs, which were divided in 6 groups, each containing 7 animals. The pigs were 3-4 weeks old, at the start of the study, and weighed 5-8 kg each. The description of the groups, is presented in Table 5. All animals in groups 1, 2, and 4 (intradermal immunization using microneedles) received the following treatment. The application sites were clipped of all hair and then shaved to further ensure a smooth surface. The sites were then washed with water and allowed to air dry. The application method consisted of applying 2 adhesive patches, each containing a coated microneedle array, to a pretreated spot on the pig's ear and pressing them for 1 minute, ensuring insertion. The patch was allowed then to remain in place, undisturbed, for 29 additional minutes. The patches were then removed and shipped back for analysis, which showed practically complete elimination of the coating in the application process. Other groups received no special treatment. Each animal in Groups 3 and 6 (intramuscular administration) received a 1 mL injection of 20 μg/mL HBsAg (i.e. 20 μg dose) liquid formulation in the neck, behind the ears. Each animal in Group 5 (intradermal administration) received four 50 μL injections of liquid formulation in four different spots on the ear, for a total of 200 μL of 100 μg/mL HBsAg solution (i.e. 20 μg dose). All subjects were anesthetized during the immunization with a combination of Xylazine and Ketamine. The blood samples were collected prior to being immunized (0 weeks) and then at 2 and 4 weeks after being immunized.

Antigen-specific antibodies (IgG) in pig sera were determined by ELISA. Immulon2 96U microtiter plates were coated with HbsAg (Fitzgerald Industries International, Inc.) at 5 ug/ml in ELISA coating buffer, pH 9.6 and incubated overnight at 4° C. The plates were washed six times with TBS containing 0.05% Tween 20 (TBST). Four-fold serial dilutions of sera in TBST starting from 1/10 were added to the wells and the plate was incubated 2 hours at room temperature or overnight at 4° C. along with positive and negative serum. Unbound serum was removed by washing the plates six times with TBST. KPL Goat anti-Swine IgG (H+L) alkaline phosphatase labeled affinity purified antibodies (Invitrogen) (diluted 1/5000) was added and the plates were incubated for 1 hour at room temperature. The plates were washed six times with TBST and HBsAg specific IgG antibodies were detected by adding 1 mg/mL of p-nitrophenyl phosphate di(Tris) salt in 1% Diethanolamine—0.5 mM magnesium chloride buffer, pH 9.8. The reaction was allowed to run for two hours and the absorbance was measured at A 405 nm, reference λ490 nm, using Benchmark™M Microplate Reader (Bio-Rad Laboratories, Hercules, Calif.). The titers were calculated as the reciprocal of the highest sample dilution producing a signal identical to that of the negative sample at the starting dilution plus three times standard deviation. The average antibody titers for groups I-VI were calculated and plotted using Microsoft Excel.

TABLE 5
Description of Animal Experiments

| Animal | | Formulation | |
|---|---|---|---|
| Group | | from | Dose |
| Number | Description | Example | μg |
| 1 | Microneedles containing HBsAg and | 1 | 20* |
| | PCPP | | |
| 2 | Microneedles containing low dose | 2 | 10* |
| | HBsAg and PCPP | | |
| 3 | Intramuscular Injection of HBsAg | 3 | 20 |
| | and PCPP | | |
| 4 | Microneedles containing HBsAg | 4 | 20* |
| 5 | Intradermal Injection of HBsAg | 5 | 20 |
| 6 | Intramuscular Injection of HBsAg | 6 | 20 |

*Antigen doses on microneedles were evaluated and rounded based on the analysis of representative arrays as described in examples 2, 3 and 5 and Table 4. 2 arrays of microneedles per animal were used in the study.

The results of the study are presented in FIG. 13. As seen from the Figure, the immune responses for both groups, which received HBsAg formulated with PCPP (Group 1 and 2), intradermally using microneedles, were superior to those in all other groups. In fact, serum IgG specific HBsAg titers for these formulations are approximately 1 order of magnitude higher than those induced by formulation of HBsAg and PCPP administered intramuscularly (Group 3), and approximately 2 orders of magnitude higher than those induced by microneedle formulations containing approximately the same dose of HBsAg (Group 4) or both intramuscular and intradermal formulations of solution formulations of HBsAg (Groups 5 and 6).

Although it is already known, that PCPP is a potent adjuvant for intramuscularly administered vaccine formulations, i.e. capable of enhancing the immune response when co-administered with the antigen, such effect is clearly limited as can be seen from the comparison of immune responses for Groups 3 and 6 (FIG. 13). In fact, comparison of results for Groups 1 and 4 (intradermal adjuvant effect of PCPP) as opposed to Groups 3 and 6 (intramuscular adjuvant effect of PCPP) leads to a conclusion that intradermal adjuvant effect of PCPP appears to be at least 10 times higher (4 week datapoint). In fact, based on additive immune responses for Group 3 (HBsAg adjuvanted with PCPP, intramuscular administration) and Group 4 (intradermal microneedle administration, no PCPP) it is difficult to anticipate the results for Group 1 combining both microneedle administration and PCPP. Such synergistic effect of intradermal administration and PCPP adjuvant, suggests superior immunostimulating potency of polyphosphazene adjuvants as intradermal immunoadjuvants, as opposed to the effect already known for intramuscular administration of the same adjuvants.

The disclosures of all patents, publications (including published patent applications), depository accession numbers, and database accession numbers are hereby incorporated by reference to the same extent as if each patent, publication, depository accession number, and database accession number were specifically and individually incorporated by reference.

It is to be understood, however, that the scope of the present invention is not to be limited to the specific embodiments described hereinabove. The invention may be practiced other than as particularly described and still be within the scope of the accompanying claims.

What is claimed is:

1. An intradermally administered pharmaceutical product for producing an immune response in a human or in an animal comprising: an antigen and a polyphosphazene polyelectrolyte adjuvant in an amount effective to elicit an immune response in the human or in the animal against said antigen.

2. The pharmaceutical product of claim 1, wherein said polyphosphazene polyelectrolyte adjuvant is poly[di(carboxylatophenoxy)phosphazene].

3. The pharmaceutical product of claim 1, wherein said product further comprises at least one microneedle.

4. The pharmaceutical product of claim 3, wherein said microneedle is a solid microneedle.

5. The pharmaceutical product of claim 3, wherein said microneedle is a microneedle, coated with a solid formulation containing an antigen and a polyphosphazene polyelectrolyte adjuvant.

6. The pharmaceutical product of claim 5 wherein said polyphosphazene polyelectrolyte adjuvant is poly[di(carboxylatophenoxy)phosphazene].

7. The pharmaceutical product of claim 3, wherein said microneedle is a microneedle, microfabricated using formulation containing an antigen and a polyphosphazene polyelectrolyte adjuvant.

8. The pharmaceutical product of claim 7 wherein said polyphosphazene polyelectrolyte adjuvant is poly[di(carboxylatophenoxy)phosphazene].

9. The pharmaceutical product of claim 3, wherein said microneedle is a hollow microneedle.

10. The pharmaceutical product of claim 2, wherein said product further comprises at least one microneedle.

11. The pharmaceutical product of claim 6, wherein said microneedle is a solid microneedle.

12. The pharmaceutical product of claim 6, wherein said microneedle is a hollow microneedle.

13. A method for producing an immune response in a human or in an animal comprising: producing an immune response in a human or in an animal by intradermally administering to the human or to the animal an antigen and a polyphosphazene polyelectrolyte adjuvant in an amount effective to elicit an immune response in the human or in the animal against said antigen.

14. The method of claim 13, wherein said polyphosphazene polyelectrolyte adjuvant is poly[di(carboxylatophenoxy)phosphazene].

15. The method of claim 13, wherein said intradermally administering of said antigen and said polyphosphazene polyelectrolyte adjuvant is effected by the use of at least one microneedle.

16. The method of claim 13, wherein said microneedle is a solid microneedle.

17. The method of claim 15, wherein said microneedle is a microneedle, coated with a solid formulation containing an antigen and a polyphosphazene polyelectrolyte adjuvant.

18. The method of claim 17 wherein said polyphosphazene polyelectrolyte adjuvant is poly[di(carboxylatophenoxy)phosphazene].

19. The method of claim 15, wherein said microneedle is a microneedle, microfabricated using formulation containing said antigen and said polyphosphazene polyelectrolyte adjuvant.

20. The method of claim 19 wherein said polyphosphazene polyelectrolyte adjuvant is poly[di(carboxylatophenoxy)phosphazene].

21. The method of claim 15, wherein said microneedle is a hollow microneedle.

22. The method of claim 14, wherein said product further comprises at least one microneedle.

23. The method of claim 18, wherein said microneedle is a solid microneedle.

24. The method of claim 18, wherein said microneedle is a hollow microneedle.
